(19) United States
(12) Patent Application Publication
(10) Pub. No.: US 2024/0173129 A1
(43) Pub. Date: May 30, 2024

(54) SKIRT FOR PROSTHETIC HEART VALVE

(71) Applicant: EDWARDS LIFESCIENCES CORPORATION, Irvine, CA (US)

(72) Inventors: Noam Nir, Pardes-Hanna (IL); Michael Bukin, Pardes Hanna (IL)

(21) Appl. No.: 18/519,557

(22) Filed: Nov. 27, 2023

Related U.S. Application Data
(63) Continuation of application No. PCT/US22/32053, filed on Jun. 3, 2022.
(60) Provisional application No. 63/196,529, filed on Jun. 3, 2021.

(51) Int. Cl. A61F 2/24

(52) U.S. Cl. CPC A61F 2/2418; A61F 2/243; A61F 2002/9511; A61F 2250/0071

(57) ABSTRACT

A skirt for an implantable prosthetic device can include an annular fabric matrix radially expandable from a radially compressed configuration to a first diameter upon application of a radially outwardly directed force via the implantable prosthetic device, and a plurality of frangible restriction filaments embedded in the fabric matrix. Each restriction filament can have a selected maximum diameter different from that of at least one of the other restriction filaments, and each restriction filament can break when a radially outwardly directed force applied to the restriction filament exceeds a predetermined threshold.

### CROSS REFERENCE TO RELATED APPLICATIONS

This application is a continuation of International Application No. PCT/US2022/032053, filed on Jun. 3, 2022, which claims the benefit of U.S. Application No. 63/196,529, filed on Jun. 3, 2021, both of which are incorporated herein by reference in their entirety.

### FIELD

The present disclosure relates to implantable, mechanically expandable prosthetic devices, such as prosthetic heart valves, and to methods and delivery assemblies for, and including, such prosthetic devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Prosthetic heart valves that rely on a mechanical actuator for expansion can be referred to as “mechanically expandable” prosthetic heart valves. Mechanically expandable prosthetic heart valves can provide one or more advantages over self-expandable and balloon-expandable prosthetic heart valves. For example, mechanically expandable prosthetic heart valves can be expanded to various diameters. Mechanically expandable prosthetic heart valves can also be compressed after an initial expansion (e.g., for repositioning and/or retrieval).

When deploying a prosthetic valve, it is important to avoid exerting excessive radial force on the native annulus of the patient, which can rupture the native heart valve annulus. To avoid damage to the native tissue, it is desirable to monitor the diameter of the prosthetic valve and/or the radial force exerted by the prosthetic valve during deployment.

Unfortunately, known methods for measuring diameter and radial force suffer from several problems. For example, measurement methods relying on measuring the displacement of an actuation mechanism fail to account for factors such as compression of the delivery device and/or elongation of the actuation mechanism under tension. Thus, despite the recent advancements in percutaneous valve technology, there remains a need for improved devices and methods for monitoring the diameter and radial force of transcatheter heart valves during implantation.

### SUMMARY

In a representative example, a skirt for an implantable prosthetic device can comprise an annular fabric matrix radially expandable from a radially compressed configuration to a first diameter upon application of a radially outwardly directed force via the implantable prosthetic device, and a plurality of frangible restriction filaments embedded in the fabric matrix. Each restriction filament can have a selected maximum diameter different from that of at least one of the other restriction filaments, and each restriction filament being configured to break when a radially outwardly directed force applied to the restriction filament exceeds a predetermined threshold.

In another representative example, a skirt for an implantable prosthetic device can comprise an annular body configured to extend around a circumference of the prosthetic device, and one or more sets of frangible restriction filaments coupled to the annular body. The annular body can be radially expandable from a radially compressed configuration to a first diameter upon application of a radially outwardly directed force via the implantable prosthetic device. A first set of frangible restriction filaments of the one or more sets of frangible restriction filaments can be configured to break when the radially outwardly directed force exceeds a first predetermined threshold to allow radial expansion of the annular body to a second diameter, and the one or more set of frangible restriction filaments can remain embedded within the annular body when broken.

In a representative example, an assembly can comprise an implantable prosthetic device comprising a frame movable between a radially compressed configuration and a radially expanded configuration, and a skirt. The skirt can comprise an annular fabric matrix radially expandable from a radially compressed configuration to a first diameter upon application of a radially outwardly directed force via the implantable prosthetic device, and at least one first frangible restriction filament and at least one second frangible restriction filament woven in the fabric matrix, wherein the first frangible restriction filament has a first maximum diameter and the second frangible restriction filament has a second maximum diameter that is greater than the first maximum diameter. The first frangible restriction filament can be configured to break when the frame is expanded to the first maximum diameter and applies a force to the first frangible restriction filament sufficient to break the first frangible restriction filament. The second frangible restriction filament can be configured to break when the frame is expanded to the second maximum diameter and applies a force to the second frangible restriction filament sufficient to break the second frangible restriction filament.

In a representative example, a method can comprise advancing an implantable prosthetic device comprising a skirt through a patient's vasculature to a selected implantation site, the skirt extending around a circumference of the prosthetic device and comprising an annular body and first and second restriction filaments embedded within the annular body. The method can further comprise radially expanding the prosthetic device and the skirt to a first diameter defined by the first restriction filament, applying a first expansion force greater than a first predetermined threshold to the prosthetic device to break the first restriction filament, and radially expanding the prosthetic device and the skirt to a second diameter defined by the second restriction filament.

The foregoing and other objects, features, and advantages of the disclosure will become more apparent from the following detailed description, which proceeds with reference to the accompanying figures.

### BRIEF DESCRIPTION OF THE DRAWINGS

FIG. 1 is a perspective view of a prosthetic heart valve, according to one example.

FIG. 2A is a side elevation view of the frame of the prosthetic heart valve of FIG. 1, shown in a radially compressed state.

FIG. 2B is a side elevation view of the frame of the prosthetic heart valve of FIG. 1, shown in a radially expanded state.

FIG. 3 is a perspective view of a prosthetic valve frame, shown in a radially collapsed state, having a plurality of expansion and locking mechanisms, according to another example.

FIG. 4 is a perspective view of the frame and the expansion and locking mechanisms of FIG. 3, with the frame shown in a radially expanded state.

FIG. 5A is a perspective view of a screw of one of the expansion and locking mechanisms of FIG. 3.

FIG. 5B is a perspective view of one of the expansion and locking mechanisms of FIG. 3.

FIG. 5C is another perspective view of the frame and the expansion and locking mechanisms of FIG. 3, with the frame shown in a radially expanded state.

FIG. 6 is another perspective view of one of the expansion and locking mechanisms of FIG. 3.

FIG. 7 shows a cross sectional view of one of the expansion and locking mechanisms of FIG. 3 along with a portion of the frame.

FIG. 8 is a side elevational view of a frame for a prosthetic heart valve including an exemplary example of a valve belt shown in a radially expanded configuration.

FIG. 9 is a side elevational view of the frame and belt of FIG. 8 shown in a radially compressed configuration.

FIG. 10 is a side elevational view of a portion of the valve belt of FIG. 8 shown in a radially expanded configuration.

FIG. 11 is a side elevational view of a portion of the valve belt of FIG. 8 shown in a radially compressed configuration.

FIG. 12 is a side elevational view of a portion of an exemplary example of a valve belt.

FIG. 13 is a perspective view of a portion of an exemplary example of a valve belt.

FIG. 14 is a side elevational view of a portion of an exemplary example of a valve belt shown in a radially expanded configuration.

FIG. 15 is a side elevational view of a delivery apparatus for a prosthetic heart valve, according to one example.

FIG. 16 is a perspective view of a frame for a prosthetic heart valve including an exemplary example of a restriction band with the frame shown in a radially expanded configuration.

FIG. 17 is a perspective of a prosthetic heart valve comprising the frame and restriction band of FIG. 16.

FIG. 18 is a perspective view of the frame of FIG. 16 including an exemplary example of a restriction belt with the frame shown in a radially expanded configuration.

FIG. 19 is a perspective view of the frame of FIG. 16 including an exemplary example of a plurality of restriction belts with the frame shown in a radially expanded configuration.

FIG. 20 is a perspective view of a frame for a prosthetic heart valve including an exemplary example of a helical tension member with the frame shown in a partially radially expanded configuration.

FIG. 21 is a perspective view of the frame and helical tension member of FIG. 20 with the frame shown in the radially expanded configuration.

FIG. 22 is a side elevational view of a frame for a prosthetic heart valve including an exemplary example of a skirt shown in a radially expanded configuration.

FIG. 23 is an enlarged view of a portion of the skirt of FIG. 22.

FIG. 24 illustrates a schematic view of restriction filaments of the skirt of FIG. 22.

FIG. 25 illustrates a side elevational view of an exemplary prosthetic heart valve shown in a radially expanded configuration and including an exemplary example of a skirt.

### DETAILED DESCRIPTION

### General Considerations

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

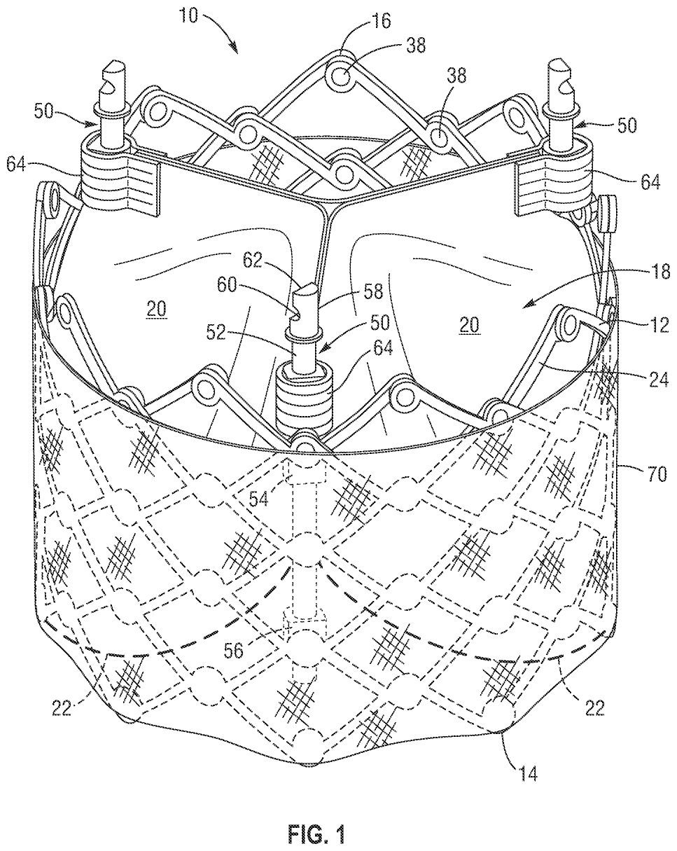

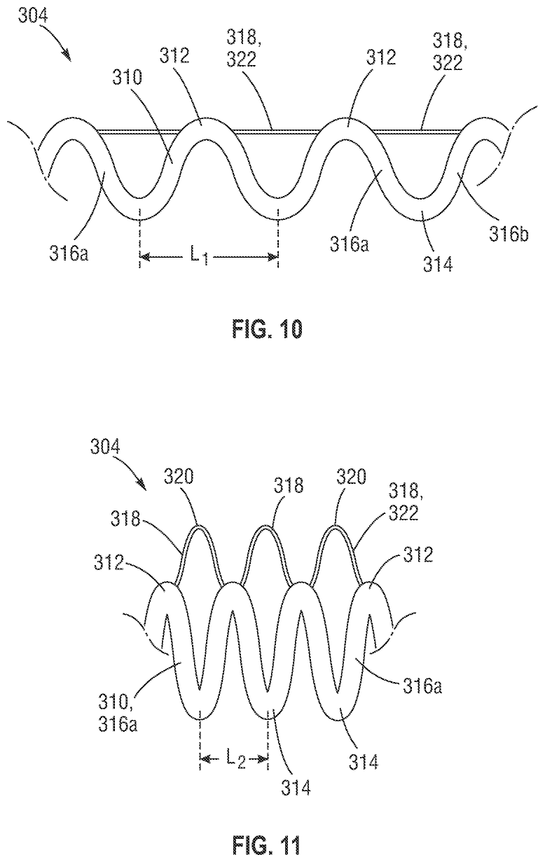

All features described herein are independent of one another and, except where structurally impossible, can be used in combination with any other feature described herein. For example, a belt 304 as shown in FIGS. 10-11 can be used in combination with prosthetic valve 10. In another example, a belt 400 as shown in FIG. 13 can be used in combination with the prosthetic valve 10 shown in FIG. 1.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

### Examples of the Disclosed Technology

Described herein are examples of prosthetic implant delivery assemblies and components thereof which can improve a physician's ability to control the size of a mechanically-expandable prosthetic implant, such as prosthetic valves (e.g., prosthetic heart valves or venous valves), stents, or grafts, as well as facilitate separation of the prosthetic implant from the delivery assembly, during the implantation procedure. The present disclosure also provides belts for use with such prosthetic implants. The belts can comprise frangible portions configured to break when a predetermined force is applied, thus allowing the force exerted by the prosthetic implant on the native anatomy to be calculated.

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the valves disclosed herein may be used with a variety of implant delivery apparatuses, and examples thereof will be discussed in more detail later.

FIG. 1 shows an exemplary prosthetic valve 10, according to one example. The prosthetic valve 10 can include an annular stent or frame 12 having an inflow end 14 and an outflow end 16. The prosthetic valve 10 can also include a valvular structure 18 which is coupled to and supported inside of the frame 12. The valvular structure 18 is configured to regulate the flow of blood through the prosthetic valve 10 from the inflow end 14 to the outflow end 16.

The valvular structure 18 can include, for example, a leaflet assembly comprising one or more leaflets 20 made of a flexible material. The leaflets 20 can be made from in whole or part, biological material, bio-compatible synthetic materials, or other such materials. Suitable biological material can include, for example, bovine pericardium (or pericardium from other sources). The leaflets 20 can be secured to one another at their adjacent sides to form commissures, each of which can be secured to a respective actuator 50 or the frame 102.

In the depicted example, the valvular structure 18 comprises three leaflets 20, which can be arranged to collapse in a tricuspid arrangement. Each leaflet 20 can have an inflow edge portion 22. As shown in FIG. 1, the inflow edge portions 22 of the leaflets 20 can define an undulating, curved scallop shape that follows or tracks a plurality of interconnected strut segments of the frame 12 in a circumferential direction when the frame 12 is in the radially expanded configuration. The inflow edges of the leaflets can be referred to as a “scallop line.”

In some examples, the inflow edge portions 22 of the leaflets 20 can be sutured to adjacent struts of the frame generally along the scallop line. In other examples, the inflow edge portions 22 of the leaflets 20 can be sutured to an inner skirt, which in turn in sutured to adjacent struts of the frame. By forming the leaflets 20 with this scallop geometry, stresses on the leaflets 20 are reduced, which in turn improves durability of the valve 10. Moreover, by virtue of the scallop shape, folds and ripples at the belly of each leaflet 20 (the central region of each leaflet), which can cause early calcification in those areas, can be eliminated or at least minimized. The scallop geometry also reduces the amount of tissue material used to form valvular structure 18, thereby allowing a smaller, more even crimped profile at the inflow end 14 of the valve 10.

Further details regarding transcatheter prosthetic heart valves, including the manner in which the valvular structure can be mounted to the frame of the prosthetic valve can be found, for example, in U.S. Pat. Nos. 6,730,118, 7,393,360, 7,510,575, 7,993,394, 8,252,202, 11,135,056, and 11,399,932, all of which are incorporated herein by reference in their entireties.

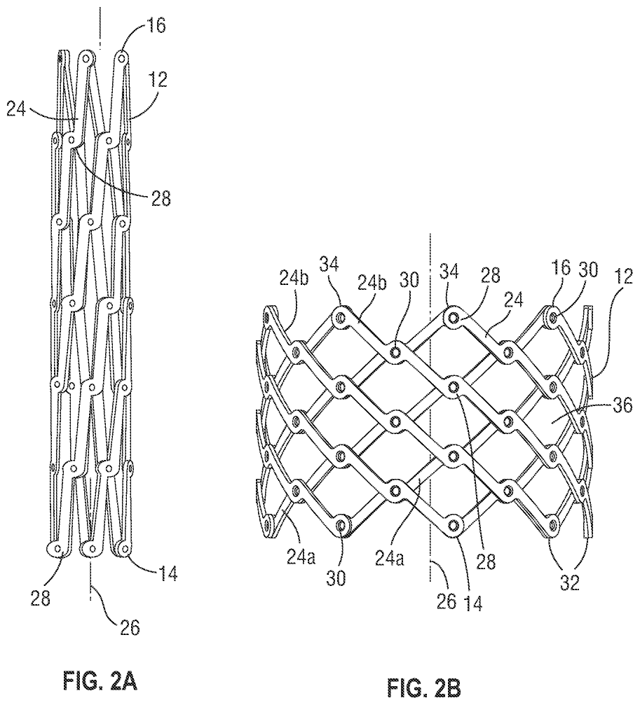

The prosthetic valve 10 can be radially compressible and expandable between a radially compressed configuration and a radially expanded configuration. FIGS. 2A-2B show the bare frame 12 of the prosthetic valve 10 (without the leaflets and other components) for purposes of illustrating expansion of the prosthetic valve 10 from the radially compressed configuration (FIG. 2A) to the radially expanded configuration (FIG. 2B).

The frame 12 can include a plurality of interconnected lattice struts 24 arranged in a lattice-type pattern and forming a plurality of apices 34 at the outflow end 16 of the prosthetic valve 10. The struts 24 can also form similar apices 32 at the inflow end 14 of the prosthetic valve 10. In FIG. 2B, the struts 24 are shown as positioned diagonally, or offset at an angle relative to, and radially offset from, a longitudinal axis 26 of the prosthetic valve 10 when the prosthetic valve 10 is in the expanded configuration. In other implementations, the struts 24 can be offset by a different amount than depicted in FIG. 2B, or some or all of the struts 24 can be positioned parallel to the longitudinal axis 26 of the prosthetic valve 10.

The struts 24 can comprise a set of inner struts 24a (extending from the lower left to the upper right of the frame in FIG. 2B) and a set of outer struts 24b (extending from the upper left to the lower right of the frame in FIG. 2B) connected to the inner struts 24a. The open lattice structure of the frame 12 can define a plurality of open frame cells 36 between the struts 24.

The struts 24 can be pivotably coupled to one another at one or more pivot joints or pivot junctions 28 along the length of each strut. For example, in one example, each of the struts 24 can be formed with apertures 30 at opposing ends of the strut and apertures spaced along the length of the strut. Respective hinges can be formed at the locations where struts 24 overlap each other via fasteners 38 (FIG. 1), such as rivets or pins that extend through the apertures 30. The hinges can allow the struts 24 to pivot relative to one another as the frame 12 is radially expanded or compressed, such as during assembly, preparation, or implantation of the prosthetic valve 10.

The frame struts and the components used to form the pivot joints of the frame 12 (or any frames described below) can be made of any of various suitable materials, such as stainless steel, a cobalt chromium alloy, or a nickel titanium alloy (“NiTi”), for example Nitinol. In some examples, the frame 12 can be constructed by forming individual components (e.g., the struts and fasteners of the frame) and then mechanically assembling and connecting the individual components together. Further details regarding the construction of the frame and the prosthetic valve are described in U.S. Pat. Nos. 10,603,165, 10,869,759, 10,806,573, and 11,399,932, all of which are incorporated herein by reference.

In the illustrated example, the prosthetic valve 10 can be mechanically expanded from the radially contracted configuration to the radially expanded configuration. For example, the prosthetic valve 10 can be radially expanded by maintaining the inflow end 14 of the frame 12 at a fixed position while applying a force in the axial direction against the outflow end 16 toward the inflow end 14. Alternatively, the prosthetic valve 10 can be expanded by applying an axial force against the inflow end 14 while maintaining the outflow end 16 at a fixed position, or by applying opposing axial forces to the inflow and outflow ends 14, 16, respectively.

As shown in FIG. 1, the prosthetic valve 10 can include one or more actuators 50 mounted to and equally spaced around the inner surface of the frame 12. Each of the actuators 50 can be configured to form a releasable connection with one or more respective actuators of a delivery apparatus.

In the illustrated example, expansion and compression forces can be applied to the frame by the actuators 50. Referring again to FIG. 1, each of the actuators 50 can comprise a screw or threaded rod 52, a first anchor in the form of a cylinder or sleeve 54, and a second anchor in the form of a threaded nut 56. The rod 52 extends through the sleeve 54 and the nut 56. The sleeve 54 can be secured to the frame 12, such as with a fastener 38 that forms a hinge at the junction between two struts. Each actuator 50 is configured to increase the distance between the attachment locations of a respective sleeve 54 and nut 56, which causes the frame 12 to elongate axially and compress radially, and to decrease the distance between the attachment locations of a respective sleeve 54 and nut 56, which causes the frame 12 to foreshorten axially and expand radially.

For example, each rod 52 can have external threads that engage internal threads of the nut 56 such that rotation of the rod causes corresponding axial movement of the nut 56 toward or away from the sleeve 54 (depending on the direction of rotation of the rod 52). This causes the hinges supporting the sleeve 54 and the nut 56 to move closer towards each other to radially expand the frame or to move farther away from each other to radially compress the frame, depending on the direction of rotation of the rod 52.

In other examples, the actuators 50 can be reciprocating type actuators configured to apply axial directed forces to the frame to produce radial expansion and compression of the frame. For example, the rod 52 of each actuator can be fixed axially relative to the nut 56 and slidable relative to the sleeve 54. Thus, in this manner, moving the rod 52 distally relative to the sleeve 54 and/or moving the sleeve 54 proximally relative to the rod 52 radially compresses the frame. Conversely, moving the rod 52 proximally relative to the sleeve 54 and/or moving the sleeve 54 distally relative to the rod 52 radially expands the frame.

When reciprocating type actuators are used, the prosthetic valve can also include one or more locking mechanisms that retain the frame in the expanded state. The locking mechanisms can be separate components that are mounted on the frame apart from the actuators, or they can be a sub-component of the actuators themselves.

Each rod 52 can include an attachment member 58 along a proximal end portion of the rod 52 configured to form a releasable connection with a corresponding actuator of a delivery apparatus. The actuator(s) of the delivery apparatus can apply forces to the rods for radially compressing or expanding the prosthetic valve 10. The attachment member 58 in the illustrated configuration comprises a notch 60 and a projection 62 that can engage a corresponding projection of an actuator of the delivery apparatus.

In the illustrated examples, the prosthetic valve 10 includes three such actuators 50, although a greater or fewer number of actuators could be used in other examples. The leaflets 20 can have commissure attachments members 64 that wrap around the sleeves 54 of the actuators 50. Further details of the actuators, locking mechanisms and delivery apparatuses for actuating the actuators can be found in U.S. Pat. Nos. 10,603,165, 10,806,573, and 11,135,056, each of which is incorporated herein by reference in its entirety. Any of the actuators and locking mechanisms disclosed in the previously filed applications can be incorporated in any of the prosthetic valves disclosed herein. Further, any of the delivery apparatuses disclosed in the previously filed applications can be used to deliver and implant any of the prosthetic valves discloses herein.

The prosthetic valve 10 can include one or more skirts or sealing members. In some examples, the prosthetic valve 10 can include an inner skirt (not shown) mounted on the inner surface of the frame. The inner skirt can function as a sealing member to prevent or decrease perivalvular leakage, to anchor the leaflets to the frame, and/or to protect the leaflets against damage caused by contact with the frame during crimping and during working cycles of the prosthetic valve. As shown in FIG. 1, the prosthetic valve 10 can also include an outer skirt 70 mounted on the outer surface of the frame 12. The outer skirt 70 can function as a sealing member for the prosthetic valve by sealing against the tissue of the native valve annulus and helping to reduce paravalvular leakage past the prosthetic valve. The inner and outer skirts can be formed from any of various suitable biocompatible materials, including any of various synthetic materials, including fabrics (e.g., polyethylene terephthalate fabric) or natural tissue (e.g., pericardial tissue). Further details regarding the use of skirts or sealing members in prosthetic valve can be found, for example, in U.S. Pat. No. 11,399,932, which is incorporated herein by reference in its entirety.

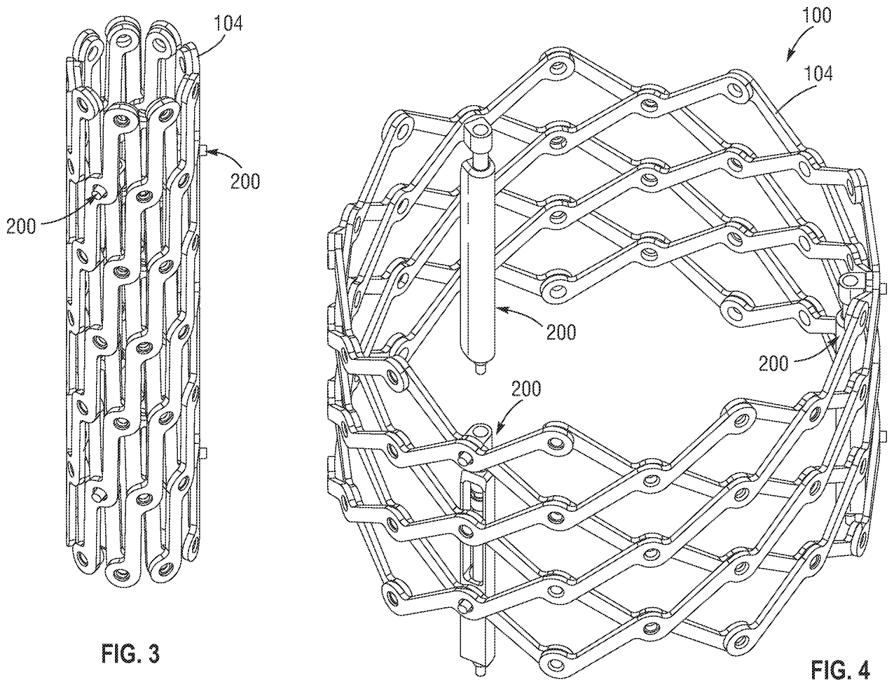

FIGS. 3-4 show another example of a prosthetic valve 100 comprising a frame 104 and expansion and locking mechanisms 200 (also referred to as “actuators”). It should be understood that the prosthetic valve 100 can include leaflets 20 and other soft components, such as one or more skirts 70, which are removed for purposes of illustration. Expansion and locking mechanism 200 can be used to both radially expand and lock the prosthetic valve in a radially expanded state. In the example of FIGS. 3 and 4, three expansion and locking mechanisms 200 are attached to the frame 104 but in other example delivery assemblies, any number of expansion and locking mechanisms 200 can be used. FIG. 3 shows the expansion and locking mechanisms 200 attached to the frame 104 when the frame is in a radially collapsed configuration and FIG. 4 shows expansion and locking mechanisms attached to the frame when the frame is in a radially expanded configuration.

It will be appreciated that prosthetic valve 100 can, in certain examples, use other mechanisms for expansion and locking, such as linear actuators, alternate locking mechanisms, and alternate expansion and locking mechanisms. Further details regarding the use of linear actuators, locking mechanisms, and expansion and locking mechanisms in prosthetic valve can be found, for example, in U.S. Pat. No. 10,603,165, which is incorporated herein by reference in its entirety.

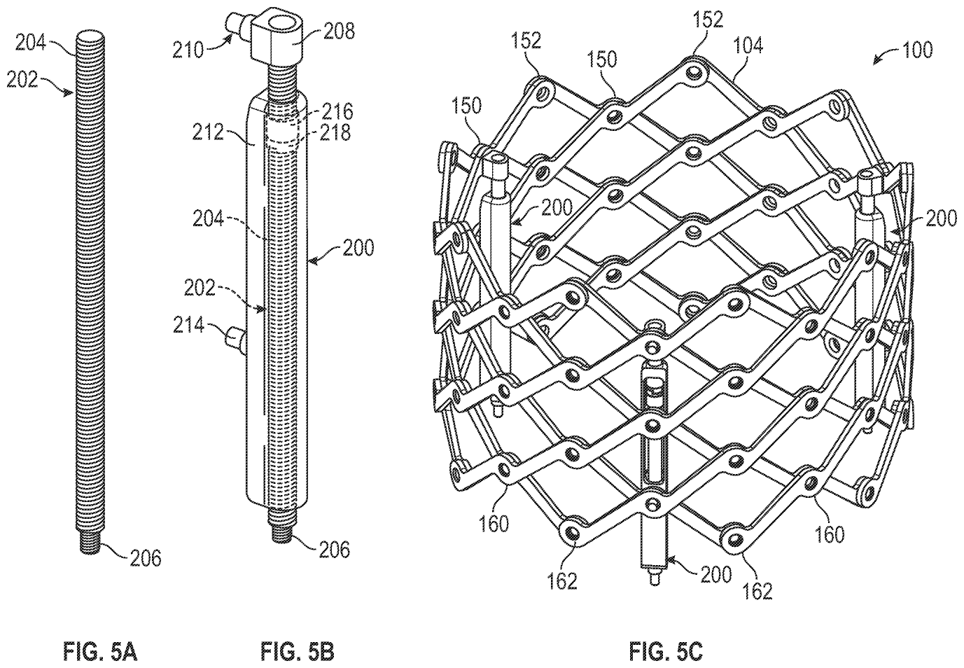

Referring to FIGS. 5A-5C, the expansion and locking mechanism 200 in the illustrated example can include an actuator screw 202 (which functions as a linear actuator or a push-pull member in the illustrated example) comprising a relatively long upper, or distal, portion 204 and a relatively shorter lower, or proximal, portion 206 at the proximal end of the screw 200, wherein the lower portion has a smaller diameter than the upper portion. Both the upper and lower portions 204, 206 of the screw 202 can have externally threaded surfaces.

The actuator screw 200 can have a distal attachment piece 208 attached to its distal end having a radially extending distal valve connector 210. The distal attachment piece 208 can be fixed to the screw 202 (e.g., welded together or manufactured as one piece). The distal valve connector 210 can extend through an opening at or near the distal end of the frame 104 formed at a location on the frame where two or more struts intersect as shown in FIG. 5C. The distal valve connector 210 can be fixed to the frame 104 (e.g., welded). Due to the shape of the struts, the distal end of the frame 104 comprises an alternating series of distal junctions 150 and distal apices 152. In the illustrated example, the distal valve connectors 210 of the three expansion and locking mechanisms 200 are connected to the frame 104 through distal junctions 150. In other examples, one or more distal valve connectors 210 can be connected to the frame 104 through distal apices 152. In other examples, the distal valve connectors 210 can be connected to junctions closer to the proximal end of the frame 104.

The expansion and locking mechanism 200 can further include a sleeve 212. The sleeve 212 can be positioned annularly around the distal portion 204 of the screw 202 and can contain axial openings at its proximal and distal ends through which the screw 202 can extend. The axial openings and the lumen in the sleeve 212 can have a diameter larger than the diameter of the distal portion 204 of the screw 202 such that the screw can move freely within the sleeve (the screw 202 can be moved proximally and distally relative to the sleeve 212). Because the actuator screw 202 can move freely within the sleeve, it can be used to radially expand and/or contract the frame 104 as disclosed in further detail below.

The sleeve 212 can have a proximal valve connector 214 extending radially from its outer surface. The proximal valve connector 214 can be fixed to the sleeve 212 (e.g., welded). The proximal valve connector 214 can be axially spaced from the distal valve connector 210 such that the proximal valve connector can extend through an opening at or near the proximal end of the frame 104. The proximal end of the frame 104 comprises an alternating series of proximal junctions 160 and proximal apices 162. In the illustrated example, the proximal valve connectors 214 of the three expansion and locking mechanisms 200 are connected to the frame 104 through proximal junctions 160. In other examples, one or more proximal valve connectors 214 can be connected to the frame 104 through proximal apices 162. In other examples, the proximal valve connectors 214 can be connected to junctions closer to the distal end of the frame 104.

It should be understood that the distal and proximal connectors 210, 214 need not be connected to opposite ends of the frame. The actuator 200 can be used to expand and compress the frame as long as the distal and proximal connectors are connected to respective junctions on the frame that are axially spaced from each other.

A locking nut 216 can be positioned inside of the sleeve 212 and can have an internally threaded surface that can engage the externally threaded surface of the actuator screw 202. The locking nut 216 can have a notched portion 218 at its proximal end, the purpose of which is described below. The locking nut can be used to lock the frame 104 into a particularly radially expanded state, as discussed below.

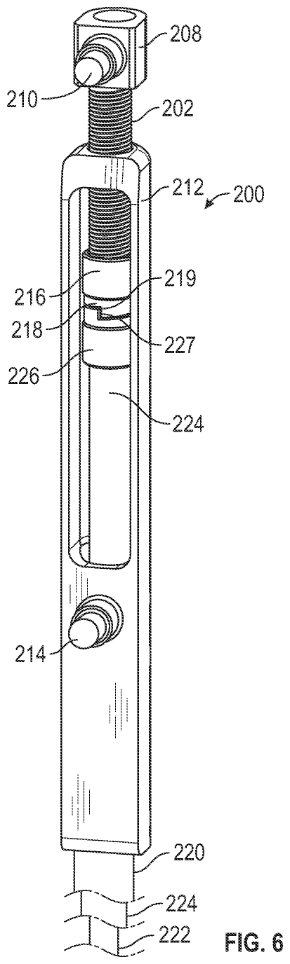

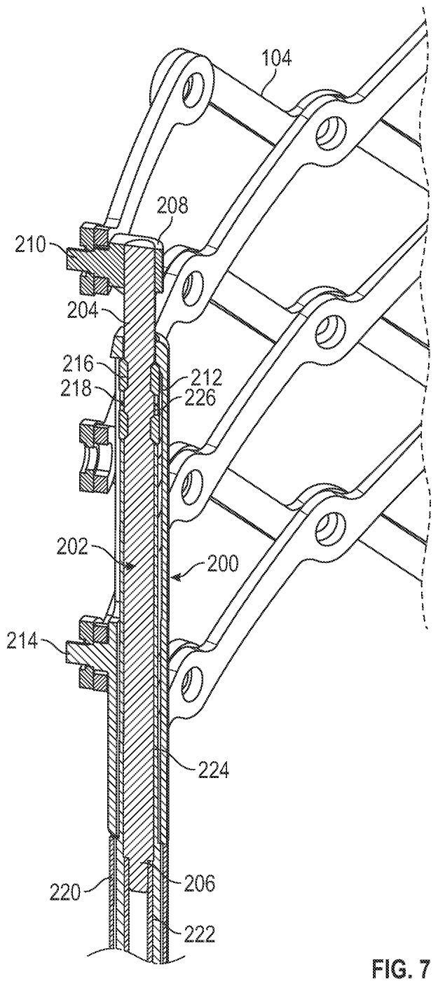

FIGS. 6 and 7 shows the expansion and locking mechanism 200 including components of a delivery apparatus not shown in FIGS. 5A-5C. As shown, the expansion and locking mechanism 200 can be releasably coupled to a support tube 220, an actuator member 222, and a locking tool 224. The proximal end of the support tube 220 can be connected to a handle or other control device (not shown) that a doctor or operator of the delivery assembly utilizes to operate the expansion and locking mechanism 200 as described herein. Similarly, the proximal ends of the actuator member 222 and the locking tool 224 can be connected to the handle.

The support tube 220 annularly surrounds a proximal portion of the locking tool 224 such that the locking tool extends through a lumen of the support tube. The support tube 220 and the sleeve are sized such that the distal end of the support tube abuts or engages the proximal end of the sleeve 212 such that the support tube is prevented from moving distally beyond the sleeve.

The actuator member 222 extends through a lumen of the locking tool 224. The actuator member 222 can be, for example, a shaft, a rod, a cable, or wire. The distal end portion of the actuator member 222 can be releasably connected to the proximal end portion 206 of the actuator screw 202. For example, the distal end portion of the actuator member 222 can have an internally threaded surface that can engage the external threads of the proximal end portion 206 of the actuator screw 202. Alternatively, the actuator member 222 can have external threads that engage an internally threaded portion of the screw 202. When the actuator member 222 is threaded onto the actuator screw 202, axial movement of the actuator member causes axial movement of the screw.

The distal portion of the locking tool 224 annularly surrounds the actuator screw 202 and extends through a lumen of the sleeve 212 and the proximal portion of the locking tool annularly surrounds the actuator member 222 and extends through a lumen of the support tube 220 to the handle of the delivery device. The locking tool 224 can have an internally threaded surface that can engage the externally threaded surface of the locking screw 202 such that clockwise or counter-clockwise rotation of the locking tool 224 causes the locking tool to advance distally or proximally along the screw, respectively.

The distal end of the locking tool 224 can comprise a notched portion 226, as can best be seen in FIG. 6. The notched portion 226 of the locking tool 224 can have an engagement surface 227 that is configured to engage a correspondingly shaped engagement surface 219 of the notched portion 218 of the locking nut 216 such that rotation of the locking tool (e.g., clockwise rotation) causes the nut 216 to rotate in the same direction (e.g., clockwise) and advance distally along the locking screw 202. The notched portions 218, 226 in the illustrated example are configured such that rotation of the locking tool 224 in the opposite direction (e.g., counter-clockwise) allows the notched portion 226 of the tool 224 to disengage the notched portion 218 of the locking nut 216; that is, rotation of the locking tool in a direction that causes the locking tool to move proximally does not cause corresponding rotation of the nut.

In alternative examples, the distal end portion of the locking tool 224 can have various other configurations adapted to engage the nut 216 and produce rotation of the nut upon rotation of the locking tool for moving the nut distally, such as any of the tool configurations described herein. In some examples, the distal end portion of the locking tool 224 can be adapted to produce rotation of the nut 216 in both directions so as move the nut distally and proximally along the locking screw 202.

In operation, prior to implantation, the actuator member 222 is screwed onto the proximal end portion 206 of the actuator screw 202 and the locking nut 216 is rotated such that it is positioned at the proximal end of the screw. The frame 104 can then be placed in a radially collapsed state and the delivery assembly can be inserted into a patient. Once the prosthetic valve is at a desired implantation site, the frame 104 can be radially expanded as described herein.

To radially expand the frame 104, the support tube 220 is held firmly against the sleeve 212. The actuator member 222 is then pulled in a proximal direction through the support tube, such as by pulling on the proximal end of the actuator member or actuating a control knob on the handle that produces proximal movement of the actuator member. Because the support tube 220 is being held against the sleeve 212, which is connected to a proximal end of the frame 104 by the proximal valve connector 214, the proximal end of the frame is prevented from moving relative to the support tube. As such, movement of the actuator member 222 in a proximal direction causes movement of the actuator screw 202 in a proximal direction (because the actuator member is threaded onto the screw), thereby causing the frame 104 to foreshorten axially and expand radially. Alternatively, the frame 104 can be expanded by moving the support tube 220 distally while holding the actuator member 222 stationary or moving the support tube distally while moving the actuator member 222 proximally.

After the frame 104 is expanded to a desired radially expanded size, the frame can be locked at this radially expanded size as described herein. Locking the frame can be achieved by rotating the locking tool 224 in a clockwise direction causing the notched portion 226 of the locking tool to engage the notched portion 218 of the locking nut 216, thereby advancing the locking nut distally along the actuator screw 202. The locking tool 224 can be so rotated until the locking nut 216 abuts an internal shoulder at the distal end of the sleeve 212 and the locking nut 216 cannot advance distally any further (see FIG. 6). This will prevent the screw 202 from advancing distally relative to the sleeve 212 and radially compressing the frame 104. However, in the illustrated example, the nut 216 and the screw 202 can still move proximally through the sleeve 212, thereby allowing additional expansion of the frame 104 either during implantation or later during a valve-in-valve procedure.

Once the frame 104 is locked in radially expanded state, the locking tool 224 can be rotated in a direction to move the locking tool proximally (e.g., in a counter-clockwise direction) to decouple the notched portion 226 from the notched portion 218 of the locking nut 216 and to unscrew the locking tool from the actuator screw 202. Additionally, the actuator member 222 can be rotated in a direction to unscrew the actuator member from the lower portion 206 of the actuator screw 202 (e.g., the actuator member 222 can be configured to disengage from the actuator screw when rotated counter-clockwise). Once the locking tool 224 and the actuator member 222 are unscrewed from the actuator screw 202, they can be removed from the patient along with the support tube 220, leaving the actuator screw and the sleeve 212 connected to the frame 104, as shown in FIG. 5C, with the frame 104 locked in a particular radially-expanded state.

In an alternative example, the locking tool 224 can be formed without internal threads that engage the external threads of the actuator screw 202, which can allow the locking tool 224 to be slid distally and proximally through the sleeve 212 and along the actuator screw 202 to engage and disengage the nut 216.

In some examples, additional designs for expansion and locking mechanisms can be used instead of the design previously described. Details on expansion and locking mechanisms can be found, for example, in U.S. Pat. No. 10,603,165, which is incorporated herein by reference in its entirety.

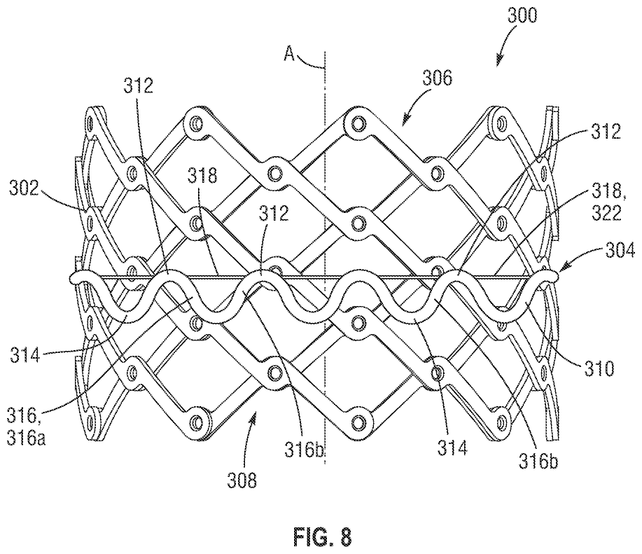

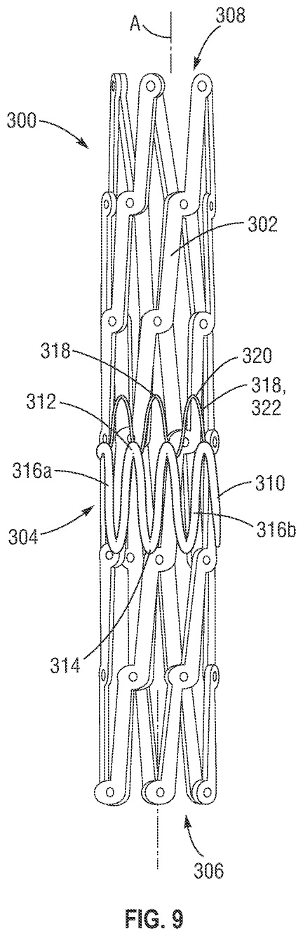

FIGS. 8-9 illustrate another exemplary example of a prosthetic heart valve 300 comprising a frame 302 and further including a belt 304 configured as a force measurement device. The prosthetic valve 300 can have an outflow end portion 306 and an inflow end portion 308 and can include a valvular structure (e.g., valvular structure 18) and inner and/or outer skirts, as previously described, although these components are omitted for purposes of illustration. The prosthetic valve 300 can also have actuators and/or locking mechanisms as previously described. While only one side of the frame 302 is depicted in FIGS. 8-9, it should be appreciated that frame 302 forms an annular structure similar to frame 12 of prosthetic valve 10 described previously, and that belt 304 has an annular shape extending around the frame 302.

The valve belts herein are described with reference to mechanically-expandable valves, such as the valves described in U.S. Pat. No. 10,603,165 and US Publication No. 2023/0225863, each of which is incorporated herein by reference. For example, some mechanical valves can comprise pivotable junctions between the struts, while others can comprise a unitary lattice frame expandable and/or compressible via mechanical means. However, it should be appreciated that the valve belts can additionally be used with other types of transcatheter prosthetic valves, including balloon-expandable prosthetic heart valves, such as disclosed in U.S. Pat. Nos. 9,393,110, and 11,096,781 and U.S. Publication No. U.S. 2019/0365530, each of which are incorporated herein by reference, and self-expandable prosthetic heart valves, such as disclosed in U.S. Pat. No. 10,098,734, which is incorporated herein by reference.

One or more selected portions of the belt 304 can be configured to rupture or break when a predetermined amount force (e.g., radial force) is applied to the belt 304 by the prosthetic valve 300, as described in more detail below. In this way, the belt 304 can be used to determine the real-time diameter of the prosthetic valve and thereby calculate the radial force applied by the prosthetic valve 300 against the surrounding tissue (e.g., the native annulus). When implanting a mechanically expandable prosthetic valve (e.g., prosthetic valve 300) it is desirable to expand the prosthetic valve to the maximum size allowed by the patient's anatomical considerations while mitigating the risk of annular rupture (e.g., by selecting a size similar to the native annulus). To ensure optimal implantation size, the diameter of the prosthetic valve and the radial force applied to the annulus by the prosthetic valve can be monitored in real time during the implantation process using a measurement device such as belt 304.

The belt 304 can comprise a generally annular or toroidal body 310 extending around the circumference of the frame 302. The body 310 can have a continuous, undulating shape comprising a plurality of peaks 312 alternating with a plurality of valleys 314 around its circumference. The peaks 312 can be positioned such that they are above the valleys 314 along a longitudinal axis A of the prosthetic valve in the orientation shown in FIG. 8. In the illustrated example, the peaks 312 point toward the outflow end 306 of the prosthetic valve 300 and the valleys 314 point toward the inflow end 308 of the prosthetic valve 300.

A plurality of struts 316 can connect adjacent peaks and/or valleys 312, 314. For example, in the illustrated example, from left to right, a first strut 316a extends from each peak 312 to each valley 314 and a second strut 316b extend up from the valley to the next peak 312. The struts 316 can be configured in a variety of shapes and sizes, e.g., straight, curved, zig-zag, symmetrical, asymmetrical, etc. For example, in the illustrated example each strut has a curved shape.

In an alternative example, the annular body 310 of the belt can be formed of strut sections coupled to one another at respective hinges where the strut sections overlap. For example, the strut sections can overlap at each peak 312 and/or valley 314 and can be coupled together via fasteners, such as rivets or pins that extend through the strut sections. The hinges can allow the strut sections to pivot relative to one another as the belt 304 is radially expanded or compressed, such as during expansion and/or compression of the prosthetic valve 300.

As shown in the illustrated example, the body 310 can be radially compressible and expandable between a radially compressed state (FIGS. 9 and 11) and a radially expanded state (FIGS. 8 and 10). Thus, the belt 304 can be crimped or retained on the radially compressed prosthetic valve in the radially compressed state during delivery, and then expanded to the radially expanded state by expansion of the prosthetic valve at the implantation site.

As shown in FIGS. 10-11, when the belt 304 is in the radially expanded position, the circumferential length between adjacent peaks 312 and/or adjacent valleys 314 can be greater than the circumferential length between adjacent peaks 312 and/or adjacent valleys 314 when in the radially compressed state. For example, in FIG. 10 the adjacent valleys 314 have a first circumferential length L₁ between them, and in FIG. 11 the adjacent valleys have a second, smaller circumferential length L₂ between them.

In particular examples, the belt 304 (including the body 310, and extension members 318 and/or frangible members 322, described below) can be made of any of the materials described above for the frame 12, including, but not limited to, any of various plastically expandable materials (e.g., stainless steel or a cobalt-chromium alloy) or self-expandable materials (e.g., Nitinol).

In some examples, the belt 304 (including the body 310, and extension members 318 and/or frangible members 322) can be made of a bioresorbable material. In such examples, after the prosthetic valve 300 has been implanted at a selected implantation site within a patient's body, the belt 304 may dissolve and/or be absorbed by the patient's body. For example, in some examples the belt 304 can comprise a bioresorbable polymer configured to dissolve over time. The resorption rate of the bioresorbable belt 304 can be controlled using a variety of parameters including the polymer material, additives, processing, etc. Exemplary bioresorbable materials include, but are not limited to, Polylactide (PLA), Poly-L-Lactide (PLLA), Polyglycolide (PGA), Poly-e-Caprolactone (PCL), Trimethylene carbonate (TMC), Poly-DL-Lactide (PDLLA), Poly-b-hydroxybutyrate (PBA), Poly-p-dioxanone (PDO), Poly-b-hydroxyproprionate (PHPA), and Poly-b-malic acid (PMLA). In other examples, the body 310 can be formed from a suitable super-elastic metal or alloy, such as Nitinol. Optionally, spring steel, a cobalt-chrome alloy such as Elgiloy®, or other such elastic metals can be utilized.

The belt 304 can further comprise a plurality of circumferentially-extending extension members 318 extending between adjacent portions of the body 310. For example, in the illustrated example, the extension members 318 extend between one or more adjacent peaks 312. However, in other examples, the extension members 318 can extend between adjacent valleys 314. In still other examples, some extension members 318 can extend between adjacent peaks 312 and other extension members 318 can extend between adjacent valleys 314.

In some examples, each pair of adjacent peaks 312 and/or adjacent valleys 314 can comprise an extension member 318 between them. For example, in the illustrated example, each pair of adjacent peaks comprises an extension member 318. However, in other examples, only selected pairs of adjacent peaks 312 and/or selected pairs of adjacent valleys 314 can comprise an extension member between them.

As shown in FIG. 10, when the belt 304 is in the radially expanded condition, the extension members 318 can extend substantially parallel to the circumference of the frame 302. When the belt 304 is in the radially compressed configuration, as shown in FIG. 11, the extension members 318 can deform axially toward the inflow and/or outflow end portions 306, 308 of the frame 302. In the illustrated example, as shown in FIG. 9, the extension members 318 deform axially toward the outflow end portion 308 of the frame 302, forming a substantially U- or V-shaped member having an apex 320.

One or more selected extension members 318 can be configured as frangible members 322. The one or more frangible members 322 can be configured to break, rupture, or otherwise deform when a force (e.g., a radial force) greater than a predetermined threshold of force is applied to the belt 304, for example, by the frame 302.

As the frame 302 moves from the radially compressed configuration (FIG. 9) to the radially expanded configuration (FIG. 10), the frame foreshortens axially and expands radially. The belt 304 expands radially with the frame from a compressed diameter until it reaches a first predetermined diameter. As the prosthetic valve 300 continues to expand (e.g., using actuators 50) the frame 302 applies an increasingly greater radial force to the belt 304. When the force exceeds a first predetermined threshold, a first frangible member 322 can break, allowing the belt 304 to radially expand to a second predetermined diameter.

The radial force exerted by the prosthetic valve 300 on the native annulus when the belt 304 is at a particular diameter can be determined based at least in part on the known mathematical relationship between the prosthetic valve's diameter and the radial force the prosthetic valve exerts. During the implantation procedure, a physician can monitor the diameter of the valve to determine when the prosthetic valve is at the diameter that best fits the native annulus and when the optimal amount of radial force is being applied by the prosthetic valve to the native annulus.

As the actuators (e.g., actuators 50) continue to expand the prosthetic valve 300, the prosthetic valve 300 continues to apply force to the belt 304, which is now expanded to the second predetermined diameter. When the force exceeds a second predetermined threshold (which can be greater than or equal to the first predetermined threshold) a second frangible member 322 can break, allowing the body 310 to radially expand to a third predetermined diameter. Each time a frangible member 322 breaks, the diameter of the belt 304 (and therefore the diameter to which the frame 302 can expand) increases. The process of expansion and breakage can continue until the prosthetic valve has reached a selected size.

The belt 304 can comprise any number of frangible members 322. For example, in some examples, all the extension members 318 are frangible members 322. In other examples, only selected extension members 318 are frangible members 322.

In some examples, each frangible member 322 can be configured to break when the same predetermined threshold of force is exceeded. In other examples, each frangible member 322 can be configured to break when a different predetermined threshold of force is exceeded. In still other examples, a first set of frangible members 322 can be configured to break when a first predetermined threshold of force is exceeded, and a second set of frangible members 322 can be configured to break when a second predetermined threshold of force is exceeded. The first predetermined threshold of force can be greater than the second predetermined threshold of force, or vice versa.

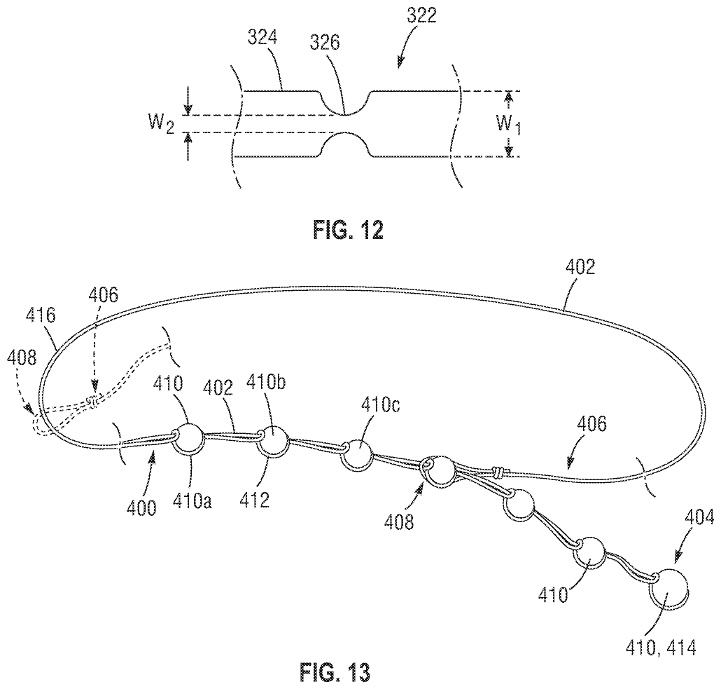

In the illustrated example, the frangible members 322 are thin wires comprising a weaker material than the material forming the body 310. In other examples, the frangible members can be, for example, cables, shafts, and/or sutures. The thin wires can be configured to break when a force greater than a predetermined threshold of force is applied. In other examples, the frangible members 322 can comprise, for example, a crack and/or a perforated region configured to make the frangible member 322 relatively weaker than the body 310. In still other examples, such as shown in FIG. 12, the frangible members 322 can comprise a strut portion 324 and a thinner portion 326 configured to break under a force exceeding a predetermined threshold. The strut portion 324 can have a first width W₁ and the thinner portion 326 can have a second width W₂ thinner than the first width.

In still other examples, when a force greater than a predetermined threshold of force is applied, the frangible members 322 can be configured to deform in a circumferential direction (e.g., by stretching) and remain permanently in the deformed configuration. For example, the frangible members 322 can be configured as springs and/or other biasing members such as deformable polymeric members.

In some examples, the frangible members 322 can comprise radiopaque portions. For example, the radiopaque portions can be disposed such that when the frangible member 322 breaks, the radiopaque portion splits into two radiopaque portions allowing a physician to determine that the frangible member 322 has been broken. For example, as the prosthetic valve is radially expanded, the physician can visualize the belt 304 using fluoroscopy. When the force applied to the belt 304 exceeds a first predetermined threshold of force, a first frangible member 322 comprising a radiopaque portion can break, splitting the radiopaque portion into two pieces. The physician can see the break in the visualization and can thus determine the amount of radial force that is being applied by the prosthetic valve to the native annulus and the diameter of the prosthetic valve. In other examples, the radiopaque portions can comprise a pattern configured to allow a physician to determine when the frangible member 322 has been broken.

In some examples, each extension member 318 can comprise a radiopaque portion. In other examples, only the frangible members 322 can comprise radiopaque portion. The radiopaque portions can comprise, for example, gold, platinum, radiopaque Nitinol, and/or combinations thereof.

In some examples, the belt 304 can comprise a cover configured to prevent the frangible members 322 from scratching and/or damaging the native annulus upon breaking. In some examples, the outer skirt of the prosthetic valve (e.g., outer skirt 70) can be configured as a cover. For example, the belt 304 can be disposed between the frame 302 and the outer skirt.

In some or all of the disclosed examples, in addition to belt 304, the prosthetic valve 300 can comprise one or more additional belts positioned at different locations along the longitudinal axis A of the prosthetic valve 300. The belts 304 can have, for example, differing predetermined diameters and/or differing predetermined thresholds for frangible member 322 breakage. In some examples, the prosthetic valve 300 can include a first belt 304 at an inflow end portion 306 of the frame and a second belt 304 at an outflow end portion 308 of the frame. In other examples, the prosthetic valve 300 can include two belts 304 positioned adjacent the inflow end portion 306 and/or the outflow end portion 308.

In a specific method for implanting a prosthetic heart valve 300 comprising one or more belts 304 in a patient's heart, a physician can use conventional techniques and/or devices to measure the approximate size of the native heart valve to facilitate selection of a desired size for the prosthetic heart valve 300. The prosthetic heart valve 300 can be mounted to the distal end portion of a delivery apparatus. The distal end portion of the delivery apparatus (along with the prosthetic valve 300) can be advanced through the patient's vasculature toward the native aortic valve. Once the prosthetic heart valve 300 is positioned at the desired implantation location (typically within the native aortic annulus), the prosthetic heart valve can be deployed (e.g., radially expanded).

To deploy the prosthetic valve, the physician can actuate the delivery apparatus, which can actuate one or more actuators (e.g., actuators 50 described above) coupled to the prosthetic valve 300. Each actuator can, for example, decrease the distance between the attachment locations of a respective sleeve and nut, causing the frame 302 to foreshorten axially and expand radially. The prosthetic valve 300 (including belt 304) can continue to expand radially until it reaches a first predetermined diameter, at which belt 304 can no longer expand. The physician can then evaluate the fit of the prosthetic valve within the native annulus and determine the force applied by the prosthetic valve 300 to the native annulus using the first predetermined diameter.

In some examples, the belt 304 can provide tactile feedback to the physician during the implantation process. For example, the physician may be able to feel (e.g., via the handle of the delivery device) when a frangible member 322 breaks.

If further expansion of the prosthetic valve is required, the prosthetic valve 300 can be expanded by continuing to actuate the actuators such that the prosthetic valve expands and applies a force to the belt 304 until a predetermined threshold of force is exceeded, causing a first frangible member 322 to break and allowing the belt 304 and therefore the prosthetic valve 300 to expand to a second predetermined diameter. The physician can then re-evaluate the fit of the prosthetic valve within the native annulus and recalculate the force applied to the native annulus. This process can be repeated as necessary until the prosthetic valve 300 is expanded to a diameter that best fits the native annulus. For example, the prosthetic valve 300 desirably is expanded to a diameter sufficient to anchor the prosthetic valve in place against the surrounding tissue with minimal or no paravalvular leakage and without over-expanding and rupturing the native annulus.

Referring now to FIG. 13, in an alternative example, the prosthetic valve 300 can comprise a belt 400. The belt 400 can be used in lieu of or in addition to belt 304, and can be disposed around a circumference of the prosthetic valve 300.

The belt 400 can comprise an elongated member 402 having a first end portion 404 and a second end portion 406 comprising a retaining member 408. The belt 400 can comprise a plurality of stoppers 410, spaced apart from each other along at least a portion of the elongated member 402. The elongated member 402 can be, for example, a cable, a wire, and/or a suture. In the illustrated example, the stoppers 410 are configured as a plurality of spheres disposed on the elongated member 402. In other examples, the stoppers 410 can have any of various shapes.

The elongated member 402 can be looped such that the first end portion 404 extends through the retaining member 408, forming an annular shape around the circumference of the prosthetic valve 300. In some examples, the elongated member 402 can include a stopper-less portion along which the retaining member 408 can slide during expansion of the prosthetic valve. The position of the stoppers 410 and the length of the stopper-less portion can be selected to provide a first diameter for the frame 302 of the prosthetic valve.

In some examples, such as the illustrated example, the stoppers 410 can be secured to the elongated member 402 at spaced apart locations using a second elongated member configured as a securing member 412. In other examples, the stoppers 410 can be formed integrally with the elongated member 402.

As shown in FIG. 13, the retaining member 408 can comprise a ring or loop formed along the end portion 406. The loop can be sized such that it has an opening equal to or slightly smaller than the diameter of a stopper 410. Accordingly, a sufficient force must be applied to force the stopper 410 to pass through the retaining member 408, as described in more detail below. In other examples, the retaining member 408 can have any of various shapes corresponding to the shape of the stoppers 410 and configured to allow a stopper 410 to pass through the retaining member 408 when a sufficient force is applied.

As the prosthetic valve 300 is expanded (increasing the circumference of the elongated member 402), the retaining member 408 can slide along the stopper-less portion 416 of the elongated member 402 until it reaches the first stopper (e.g., stopper 410a in the illustrated example). The first stopper 410a prevents the retaining member 408 from continuing to slide along the elongated member 402, thereby retaining the belt 400 (and therefore the prosthetic valve 300) at a first predetermined diameter. A first radial force exerted by the prosthetic valve 300 on the native annulus can be determined based at least in part on the known mathematical relationship between the prosthetic valve's diameter and the radial force exerted by the prosthetic valve.

As the prosthetic valve 300 continues to expand (e.g., using actuators 50) the frame 302 applies an increasingly greater force to the belt 400. When the force exceeds a first predetermined threshold the first stopper 410a can pass through the retaining member 408, allowing the retaining member to slide along the first elongated member 402 until it reaches the second stopper 410b, which holds the belt 400 at a second predetermined diameter. A second force exerted by the prosthetic valve 300 on the native annulus can be determined as described previously.

As the actuators (e.g., actuators 50 described previously) continue to expand the prosthetic valve 300, the prosthetic valve 300 will continue to apply an increasingly greater force to the belt 400. When the force exceeds a second predetermined threshold (which can be greater than or equal to the first predetermined threshold) the second stopper 410b can pass through the retaining member 408, allowing the retaining member 408 to slide along the elongated member until it reaches the third stopper 410c, which holds the belt 400 at a third predetermined diameter. Each time a stopper 410 passes through the retaining member 408, the diameter of the belt 400 (and therefore the diameter to which the frame 302 can expand) increases. The process of expansion can continue until the prosthetic valve has reached a selected size. Throughout the implantation procedure, a physician can continuously monitor the diameter of the valve to determine when the prosthetic valve is at the diameter that best fits the native annulus and when the optimal amount of radial force is being applied by the prosthetic valve to the native annulus. In some examples, the belt 400 can provide tactile feedback to the physician during the implantation process. For example, the physician may be able to feel (e.g., via the handle of the delivery device) when the retaining member 408 contacts a stopper 410 and/or when the retaining member 408 moves past a stopper 410.

In some examples, the stopper 410 positioned at the end of first end portion 404 can be configured as an enlarged stopper 414. The enlarged stopper 414 can have a diameter greater the other stoppers 410 and greater than the opening of the retaining member 408 such that the enlarged stopper 414 is prevented from passing through the retaining member 408. The enlarged stopper 414 can have a diameter large enough that no amount of force applied by the prosthetic valve 300 is sufficient to cause the enlarged stopper to pass through the retaining member 408. In this way, the enlarged stopper 414 can create a maximum diameter beyond which the prosthetic valve 300 is prevented from expanding.

In some examples, one or more of the stoppers 410 and/or the retaining member 408 can comprise radiopaque portions. As the prosthetic valve is radially expanded, the physician can visualize the belt 400 using fluoroscopy and can determine the diameter of the prosthetic valve 300 based on the position of the stoppers 410 relative to the retaining member 408. For example, in a particular instance, the physician can determine that two stoppers 410 have passed through the retaining member 408 and thereby determine that the prosthetic valve is expanded to a third predetermined diameter. The physician can then determine the radial force exerted by the prosthetic valve based at least in part on the known mathematical relationship between the prosthetic valve's diameter and the radial force exerted by the prosthetic valve.

In another alternative example, the belt can comprise a ratcheting mechanism. For example, an exemplary example of a belt can comprise an elongated member extending around a circumference of the prosthetic valve and having a first end portion and a second end portion. The second end portion can comprise a ratcheting mechanism. In some examples, the first end portion can comprise a plurality of teeth or other feature configured to interact with the ratcheting mechanism.

The elongated member can be looped such that the first end portion extends through the ratcheting mechanism, forming an annular shape around the circumference of the prosthetic valve. As the prosthetic valve expands, the first end portion can move relative to the ratcheting portion until a first predetermined diameter is reached, at which point the ratcheting mechanism can engage the first end portion and prevent further expansion of the prosthetic valve. The ratcheting mechanism can be configured to retain the valve at the first predetermined diameter until a force greater than a first predetermined threshold is applied to the belt. When the force applied by the prosthetic valve exceeds the first predetermined threshold the ratcheting member can allow the first end portion to move relative to the ratcheting member until a second predetermined diameter is reached. The process of expansion can continue until the prosthetic valve has reached a selected size.

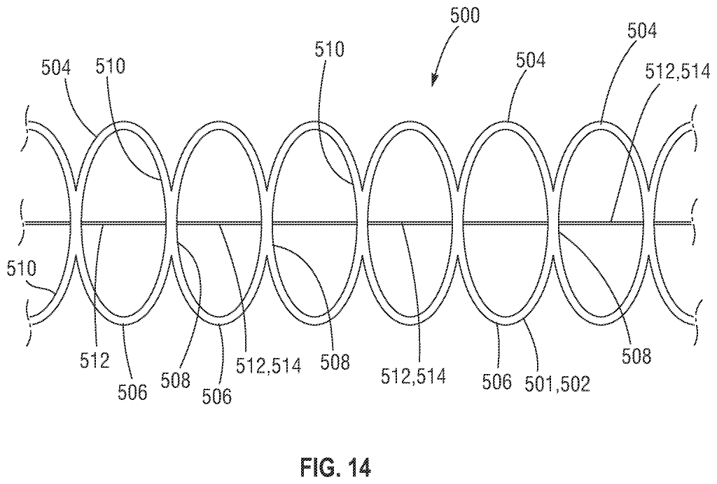

Referring now to FIG. 14, in an alternative example, the prosthetic valve 300 can comprise a belt 500. The belt 500 can be used in lieu of or in addition to belts 304 and 400, and can be disposed around a circumference of the prosthetic valve 300. Belt 500 can be similar to belt 304 except that the body 501 of belt 500 comprises a plurality of rings 502 rather than a plurality of alternating peaks and valleys.

The belt 500 can comprise an annular body portion 501 comprising a plurality of hoops or rings 502. As shown in the illustrated example, each ring 502 can have a substantially oval shape including a first end portion 504, a second end portion 506, and first and second central portions 508, 510 each extending between the first and second end portions. In other examples, the rings 502 can have any of various shapes, including but not limited to circles, squares, rectangles, triangles, diamonds, square-ovals, etc. The belt 500 can be made of any of the materials discussed above with regard to belt 304.

Each ring 502 can be coupled to one or more adjacent rings at the first and/or second central portions 508, 510. In other examples, each ring 502 can be coupled to one or more adjacent rings at the first and/or second end portions 504, 506.

The belt 500 can be radially compressible and expandable between a radially compressed state and a radially expanded state (FIG. 14). When the belt 500 is in the radially expanded position, the circumferential length between the first and second central portions 508, 510 of a respective ring 502 can be greater than the circumferential length between the first and second central portions 508, 510 of the respective ring when in the radially compressed position.

The belt 500 can comprise a plurality of circumferentially-extending extension members 512 extending between adjacent portions of the body 501. For example, in the illustrated example, the extension members 512 extend between the first and second central portions 508, 510 of each ring 502. However, in other examples, the extension members 512 can extend between the first end portions 504 of adjacent rings, between the second end portions 506 of adjacent rings, and/or any combination of the foregoing.

One or more selected extension members 512 can be configured as frangible members 514, similar to frangible members 322 described above. The one or more frangible members 514 can be configured to break, rupture, or otherwise deform, such as by stretching, when a force (e.g., a radial force) greater than a predetermined threshold of force is applied to the belt 500, for example, by the prosthetic valve 300.

As the prosthetic valve 300 expands, the belt 500 expands radially with the frame from a compressed diameter until it reaches a first predetermined diameter. As the prosthetic valve 300 continues to expand, the frame 302 applies an increasingly greater radial force to the belt 500. When the force exceeds a first predetermined threshold, a first frangible member 514 can break, allowing the belt 500 to radially expand to a second predetermined diameter. A first radial force exerted by the prosthetic valve 300 on the native annulus can be determined based at least in part on the known mathematical relationship between the prosthetic valve's diameter and the radial force exerted by the prosthetic valve.

As the prosthetic valve 300 continues to expand, the frame 302 applies an increasingly greater force to the belt 500, which is now expanded to the second predetermined diameter. When the force exceeds a second predetermined threshold (which can be greater than or equal to the first predetermined threshold) a second frangible member 514 can break, allowing the body 501 to radially expand to a third predetermined diameter. Each time a frangible member 514 breaks, the diameter of the belt 500 (and therefore the diameter to which the frame 302 can expand) increases. The process of expansion and breakage can continue until the prosthetic valve has reached a selected size.

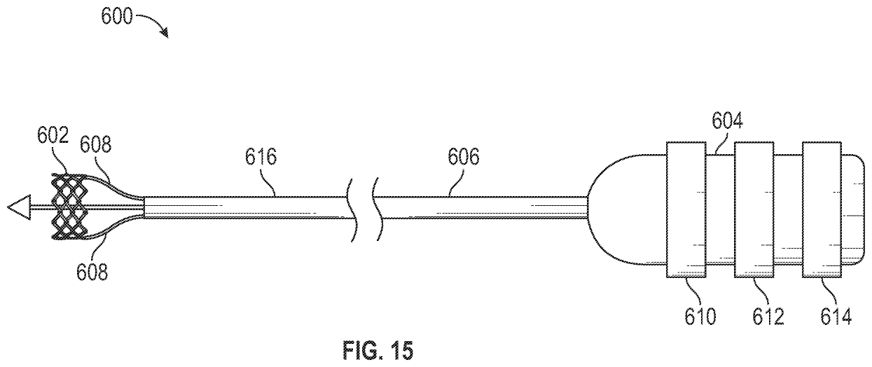

FIG. 15 illustrates a delivery apparatus 600, according to one example, adapted to deliver a prosthetic heart valve 602, such as any of the prosthetic heart valves described herein. The prosthetic valve 602 can be releasably coupled to the delivery apparatus 600. It should be understood that the delivery apparatus 600 and other delivery apparatuses disclosed herein can be used to implant prosthetic devices other than prosthetic valves, such as stents or grafts.

The delivery apparatus 600 in the illustrated example generally includes a handle 604, and a first elongated shaft 606 (which comprises an outer shaft in the illustrated example) extending distally from the handle 604.

In examples wherein the prosthetic valve 602 is a mechanically-expandable prosthetic valve, such as the example illustrated in FIG. 15, the delivery apparatus 600 can comprise at least one actuator assembly 608 extending distally through the outer shaft 606. The at least one actuator assembly 608 can be configured to radially expand and/or radially collapse the prosthetic valve 602 when actuated. Though the illustrated example shows two actuator assemblies 608 for purposes of illustration, it should be understood that one actuator 608 can be provided for each actuator on a corresponding prosthetic valve. For example, three actuator assemblies 608 can be provided for a prosthetic valve having three actuators. In other examples, a greater or fewer number of actuator assemblies can be present.

The actuator assemblies 608 can be releasably coupled to the prosthetic valve 602. For example, in the illustrated example, each actuator assembly 608 can be coupled to a respective actuator of the prosthetic valve 602. Each actuator assembly 608 can comprise, for example, a support tube, an actuator member, and a locking tool. When actuated, the actuator assembly can transmit pushing and/or pulling forces to portions of the prosthetic valve to radially expand and collapse the prosthetic valve. The actuator assemblies 608 can be at least partially disposed radially within, and extend axially through, one or more lumens of the outer shaft 606. For example, the actuator assemblies 608 can extend through a central lumen of the shaft 606 or through separate respective lumens formed in the shaft 606.

In examples wherein the prosthetic valve 602 is a self-expanding prosthetic valve or a balloon-expandable prosthetic valve, the delivery apparatus 600 need not include actuators 608.

In some examples, a distal end portion 616 of the shaft 606 can be sized to house the prosthetic valve in its radially compressed, delivery state during delivery of the prosthetic valve through the patient's vasculature. In this manner, the distal end portion 616 functions as a delivery sheath or capsule for the prosthetic valve during delivery.

The handle 602 of the delivery apparatus 600 can include one or more control mechanisms (e.g., knobs or other actuating mechanisms) for controlling different components of the delivery apparatus 600 in order to expand and/or deploy the prosthetic valve 602. For example, in the illustrated example the handle 602 comprises first, second, and third knobs 610, 612, and 614.

The first knob 610 can be a rotatable knob configured to produce axial movement of the outer shaft 606 relative to the prosthetic valve 602 in the distal and/or proximal directions in order to deploy the prosthetic valve from the delivery sheath 616 once the prosthetic valve has been advanced to a location at or adjacent the desired implantation location with the patient's body. For example, rotation of the first knob 610 in a first direction (e.g., clockwise) can retract the sheath 616 proximally relative to the prosthetic valve 602 and rotation of the first knob 610 in a second direction (e.g., counter-clockwise) can advance the sheath 616 distally. In other examples, the first knob 610 can be actuated by sliding or moving the knob 610 axially, such as pulling and/or pushing the knob. In other examples, actuation of the first knob 610 (rotation or sliding movement of the knob 610) can produce axial movement of the actuator assemblies 608 (and therefore the prosthetic valve 602) relative to the delivery sheath 616 to advance the prosthetic valve distally from the sheath 616.

In some examples, the second knob 612 can be a rotatable knob configured to produce radial expansion and/or contraction of the prosthetic valve 602. Rotation of the second knob 612 in a first direction (e.g., clockwise) can radially expand the prosthetic valve 602 and rotation of the second knob 612 in a second direction (e.g., counter-clockwise) can radially collapse the prosthetic valve 602. In other examples, the second knob 612 can be actuated by sliding or moving the knob 612 axially, such as pulling and/or pushing the knob.

In some examples, the third knob 614 can be a rotatable knob configured to retain the prosthetic heart valve 602 in its expanded configuration. Rotation of the third knob in a first direction (e.g., clockwise) can, for example, rotate each locking tool to advance the locking nuts to their distal positions to resist radial compression of the frame of the prosthetic valve. Rotation of the knob 614 in the opposite direction (e.g., counterclockwise) can rotate each locking tool in the opposite direction to decouple each locking tool from the respective nut and remove the locking tool from the respective actuator screw. In other examples, the third knob 614 can be actuated by sliding or moving the third knob 614 axially, such as pulling and/or pushing the knob.

Although not shown, the handle 604 can include a fourth rotatable knob operative connected to a proximal end portion of each actuator member. The fourth knob can be configured to rotate each actuator member, upon rotation of the knob, to unscrew each actuator member from the proximal portion of a respective actuator. Once the locking tools and the actuator members are unscrewed from the actuator screws, they can be removed from the patient along with the support tubes.

In some examples, in lieu of or in addition to a valve belt such as valve belts 304, 400, or 500, a prosthetic valve 700 having a frame 702 can comprise one or more restriction members/belts/bands 704 configured to prevent the expansion of the prosthetic valve 700 past a selected diameter. Though the illustrated prosthetic valve 700 is a self-expanding prosthetic valve, it should be understood that such restriction bands 704 can be used with any type of prosthetic valve, including mechanical valves such as disclosed in International Application No. PCT/US2021/052745 and U.S. Pat. No. 10,603,165, balloon-expandable prosthetic heart valves, such as disclosed in U.S. Pat. Nos. 9,393,110 and 11,096,781, and self-expandable prosthetic heart valves, such as disclosed in U.S. Pat. No. 10,098,734.

Prosthetic valve 700 comprises a frame 702 having an inflow end portion 706 and an outflow end portion 708, and can include a valvular structure 703 (FIG. 17) (similar to valvular structure 18) and an inner skirt 718 (FIG. 17). In some examples, the prosthetic valve 700 can further comprise one or more outer skirts, as previously described, through these components are omitted for purposes of illustration. The frame 702 can comprise a plurality of struts 710 coupled together in an open lattice pattern defining a plurality of cells 712.

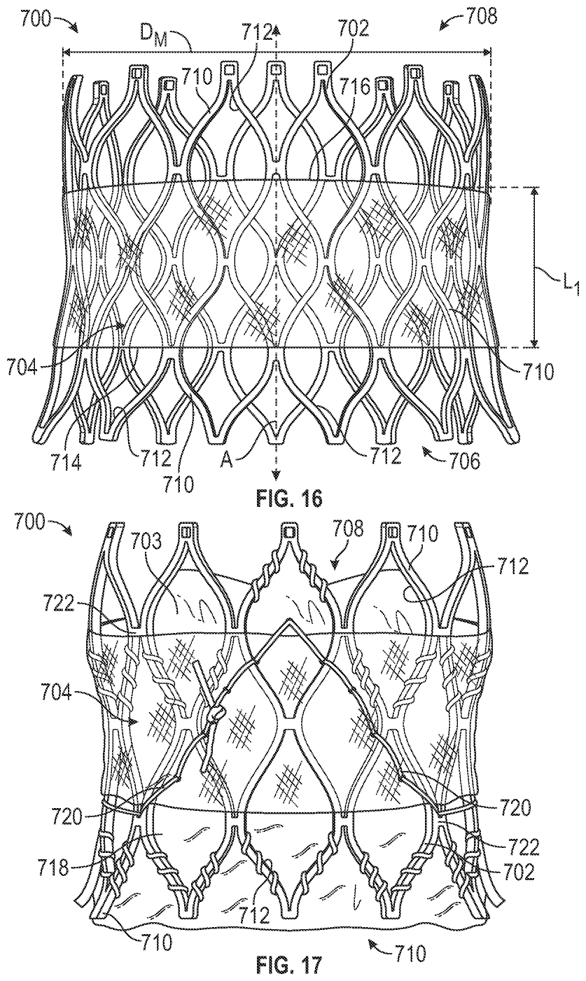

As shown in FIG. 16, the restriction band 704 can extend around a circumference of the frame 702. The restriction band 704 can be configured to expend to a maximum diameter D_{M}, such that the band allows expansion of the frame 702 to the maximum diameter D_{M} and prevents further expansion of the frame 702 past the maximum diameter D_{M}. In some examples, the restriction band 704 can comprise, for example, a resilient cloth. The restriction band 704 can have a length L₁ extending axially along a longitudinal axis A of the frame 702. In other examples, the band 704 can have a greater or shorter width. For example, in some examples, the restriction band 704 can extend along substantially the entire length of the prosthetic valve 700, thereby uniformly restricting the maximum expansion of the prosthetic valve along its length.

In some examples, the restriction band 704 can comprise fibers woven such that they extend at a non-parallel and non-perpendicular angle relative to the inflow edge 714 and/or outflow edge 716 of the band 704. For example, the fibers can be woven such that they are oriented at a 45 degree angle relative to the outflow and inflow edges 714, 716. Such a configuration allows the restriction band 704 to elongate the in the axial and/or circumferential direction with the movement of the frame 702.

As mentioned, the prosthetic valve 700 can be a self-expanding prosthetic valve. In some examples, the prosthetic valve 700 can be deployed to a diameter less than the maximum diameter D_{M} of the restriction band 704 during implantation (e.g., such that the band 704 does not restrict expansion and/or is not fully tensioned around the prosthetic valve 700). For example, the maximum diameter D_{M} can be greater than the diameter of the native annulus. In such examples, if the native annulus loses tension over time, thereby allowing prosthetic valve 700 to further self-expand, the restriction band 704 can function to prevent expansion of the prosthetic valve 700 beyond the maximum diameter D_{M} of the restriction band. Such a configuration advantageously mitigates the risk of delayed coronary obstruction (DCO) by preventing expansion past the maximum selected diameter D_{M} even if the force applied by the native annulus lessens. The force applied by the restriction band 704 at the maximum diameter D_{M} can be greater than the radial expansion force of the self-expandable valve.

In some examples, a physician may be provided with a variety of prosthetic valves each comprising a restriction band 704 having a different selected maximum diameter D_{M}. Accordingly, the physician can choose the appropriate prosthetic valve/restriction band combination having a maximum diameter D_{M} best suited to the patient's specific anatomy. In other examples, a physician can select a specific restriction band 704 to be attached to a pre-assembled prosthetic valve.

Though in the illustrated example, the restriction band 704 is positioned at a central position along a longitudinal axis A of the frame 702, in other examples, the restriction band 304 can be positioned nearer to or further from the outflow and/or inflow ends 706, 708. For example, in some examples, the prosthetic valve 700 can comprise a restriction band 704 positioned at the outflow end portion 708 of the frame 702 (e.g., defining an A-shape). Such a configuration can advantageously restrict the outflow end portion 708 from expanding and potentially obstructing the native coronaries, while still allowing the inflow end portion 706 of the prosthetic valve to expand freely to a larger diameter within the native annulus, preventing or mitigating prosthetic valve migration if the tension of the native annulus decreases over time. In another example, the prosthetic valve 700 can comprise a restriction band 704 positioned at the inflow end 706 of the frame 702 (e.g., defining a V-shape). Such a configuration can advantageously limit over-expansion of the inflow end portion 706, thereby preventing or mitigating the risk of anatomical rupture. In still other examples, the prosthetic valve 700 can comprise a restriction band at the outflow end portion 708 and the inflow end portion 706, defining a barrel-shaped frame. Such a configuration can advantageously prevent the outflow end portion 708 from obstructing the coronary ostia while also preventing over expansion of the prosthetic valve 700 against the native annulus to mitigate risks of tissue damage or conduction disturbances.

Though the illustrated example shows only one restriction band 704, in other examples, a prosthetic valve 700 can comprise a plurality of restriction bands 704. The plurality of restriction bands 704 can be disposed along the longitudinal axis A of the prosthetic valve 700 in any of various configurations. In some examples, the restriction bands 704 can be spaced apart from one another along the longitudinal axis A of the frame 702. In other examples, the restriction bands 704 can be disposed such that they fully or partially overlap one or more adjacent bands 704.

In some examples, the restriction band 704 can be coupled to the prosthetic valve 700 during assembly of the prosthetic valve. For example, in some examples, as shown in FIG. 17, the restriction band 704 can be coupled to the prosthetic valve 700 by suturing the restriction band 704 to an inner skirt 718 using one or more sutures 720. However, in other examples, the restriction band 704 can be, for example, sutured directly to the frame 702 (e.g., along struts 710 of the frame, and/or at junctions 722 of the frame) using suture loops that extend around the struts 710 and/or junctions 722 of the frame.

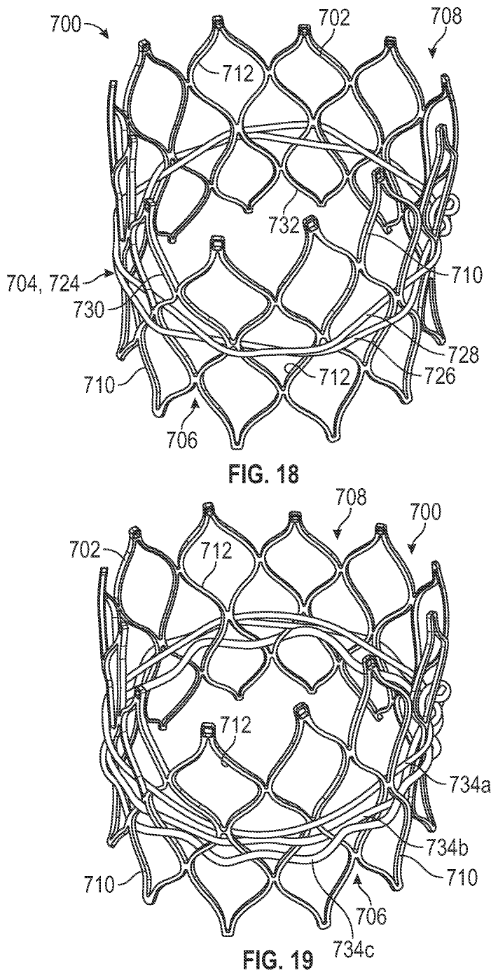

Referring to FIG. 18, in some examples, the restriction band 704 can be configured as a belt 724 comprising a wire, string, and/or cable. The belt 724 can extend circumferentially around the frame 702. In some examples, as shown in FIG. 18, the belt 724 can be coupled to the frame 702 by, for example, weaving the belt 724 through the cells 712 of the frame in an in-and-out (e.g., sinusoidal or woven) pattern. In other examples, the belt 724 can be coupled to the frame 702 via a plurality of sutures. In still other examples, the frame 702 can comprise a plurality of eyelets or apertures through which portions of the belt 724 can be threaded.

In some examples, the belt 724 can comprise multiple strands of material. For example, in the illustrated example, the belt 724 comprises a first strand 726 and a second strand 728. The first strand 726 can be woven through the cells 712 of the frame 702 in an in-and-out pattern such that portions of the first strand 726 are disposed on a radially outer surface 730 of the frame 702 and other portions of the first strand 726 are disposed on the radially inner surface 732 of the frame 702. The second strand 728 can likewise be woven through the cells 712 of the frame in an in-and-out pattern. In some examples, the second strand 728 can be woven such that, at least in parts, it opposes the first strand 726. For example, at selected circumferential points on the frame 702 the first strand 726 can be disposed on the radially outer surface 730 and the second strand 728 can be disposed on the radially inner surface 732.

Referring now to FIG. 19, in some examples, the prosthetic valve 700 can comprise a plurality of restriction belts or bands 734, each comprising a different maximum diameter D_{M} configured such that a physician can select the appropriate maximum diameter D_{M} for a patient.

As shown in FIG. 19, in some examples, the prosthetic valve 700 can comprise three restriction bands 734a, 734b, 734c. The first restriction band 734a can have a first maximum diameter D_{M1}, the second restriction band 734b can have a second maximum diameter D_{M2} greater than the first maximum diameter D_{M1}, and the third restriction band 734c can have a third maximum diameter D_{M3} greater than both the first and second maximum diameters D_{M1} and D_{M2}. In other examples, a prosthetic valve 700 can comprise a greater or fewer number of restriction bands 734.

In some examples, one or more of the restriction bands 734 can be configured as a belt comprising a wire, string, and/or cable, and/or can comprise a resilient cloth similar to restriction band 704. In some examples, each restriction band 734 can comprise the same material. However, in other examples, one or more restriction bands 734 can comprise different materials.

In use, a physician may analyze the patient's specific anatomy (e.g., using angiograms and/or CT-scans) prior to the implantation procedure, and can select an appropriate maximum diameter D_{M} for the inflow end portion and/or the outflow end portion of the frame. The physician can then cut, sever, or otherwise remove any restriction band 734 having a maximum diameter D_{M} less than that of the selected maximum diameter. Accordingly, once the prosthetic valve has been delivered to the implantation site using a delivery apparatus such as delivery apparatus 600 and the distal end portion 616 has been retracted, the inflow and/or outflow end portion of the prosthetic valve 700 can self-expand to a diameter not greater than the selected maximum diameter.

In some examples, the prosthetic valve 700 can comprise a plurality of restriction bands 734 positioned at the outflow end portion 708 and a plurality of restriction bands 734 positioned at the inflow end portion 706. By cutting any restriction bands 734 having a maximum diameter less than the selected maximum diameter, the physician can select a maximum diameter (e.g., based on the patient's specific anatomy as determined by the angiograms and/or CT-scans) for each the inflow end portion 706 and the outflow end portion 708 of the prosthetic valve. Such a configuration can advantageously prevent the outflow end portion 708 of the prosthetic valve 700 from obstructing the coronary ostia while also preventing over expansion of the prosthetic valve 700 against the native annulus to mitigate risks of tissue damage or conduction disturbances. In some examples, the physician can select restriction bands having the same maximum diameter for each the inflow and outflow end portions (e.g., to define a cylindrical or barrel-shape for the frame), however, in other examples, the physician can select restriction bands having different maximum diameters for each the inflow and outflow end portions (e.g., to define an A-shape or a V-shape for the frame).

In other examples, the restriction band 734 can be a single band that has discrete release levels (e.g., similar to belts 304, 400, and 500 described previously). One or more of such restriction bands 734 can be disposed around the frame 702. For example, a first band can be positioned at an inflow end portion and a second band can be positioned at an outflow end portion. In such examples, the physician may analyze the patient's specific anatomy as described previously to determine a selected inflow end diameter and a selected outflow end diameter for the prosthetic valve. So determined, the physician can partially release the first and second restriction bands (e.g., by severing or breaking portions of the bands, and/or by using a ratcheting mechanism) such that each band can expand to the selected maximum diameter. In some examples, the physician can release the restriction bands such that the same maximum diameter is selected for each the inflow and outflow end portions (e.g., to define a cylindrical or barrel-shape for the frame), however, in other examples, the physician can release the restriction bands such that they have different maximum diameters for each the inflow and outflow end portions (e.g., to define an A-shape or a V-shape for the frame).

The restriction band examples described herein advantageously improve usability of the prosthetic valves by providing a wide range of potential maximum diameters to which a prosthetic valve can be expanded without requiring the physician to stock and maintain a wide variety of prosthetic valves. When mechanically-expandable valves are used, a physician cannot always determine the diameter of the prosthetic valve in real time during the expansion process. Use of one or more restriction bands, such as bands 704, 724, and/or 734, can prevent inadvertent over-expansion of the mechanically expandable prosthetic valve past the selected maximum diameter. Likewise, when balloon-expandable valves are used, use of one or more restriction bands can prevent inadvertent over-expansion of the balloon-expandable prosthetic valve past the selected maximum diameter.

In some examples, such as shown in FIG. 19, each restriction band 734 of the plurality of restriction bands can be coupled to the same portion of the prosthetic valve 700 (for example, the central portion, the outflow end portion, and/or the inflow end portion) and can be spaced axially in close proximity to the other restriction bands. The space between each band and the one or more adjacent bands 734 can be selected to allow easy cutting/removal of the un-selected band(s) (e.g., using scissors).

In the illustrated example, the restriction bands 734 are coupled to the prosthetic valve 700 by weaving the restriction bands 734 through the cells 712 of the frame 702 in an in-and-out pattern. To remove any undesired restriction bands 734, the physician can cut through the band itself. In other examples, each of the plurality of restriction bands 734 can be coupled to prosthetic valve 700 at a plurality of discrete attachment points using, for example, suture loops. For example, a restriction band 734 can be coupled to the prosthetic valve 700 using two circumferentially opposing suture loops. In such examples, a physician can cut the two suture loops in order to remove the restriction band 734.

In some examples, the one or more restriction bands can be configured as filaments embedded within a fabric matrix, for example, in a skirt of the prosthetic valve (e.g., such as an inner skirt or an outer skirt as described previously). The fabric matrix can comprise a woven fabric comprising, for example, a plain weave formed from a plurality of warp and weft filaments or yarns. Additional filaments or yarns can be embedded or woven into the fabric, wherein the additional filaments or yarns are configured as restriction bands or restriction members. For example, the skirt can comprise a material (e.g., a woven cloth or fabric) having a plurality of filament groups each configured to restrict the prosthetic valve to a selected diameter unless and until a force above a selected threshold is applied.

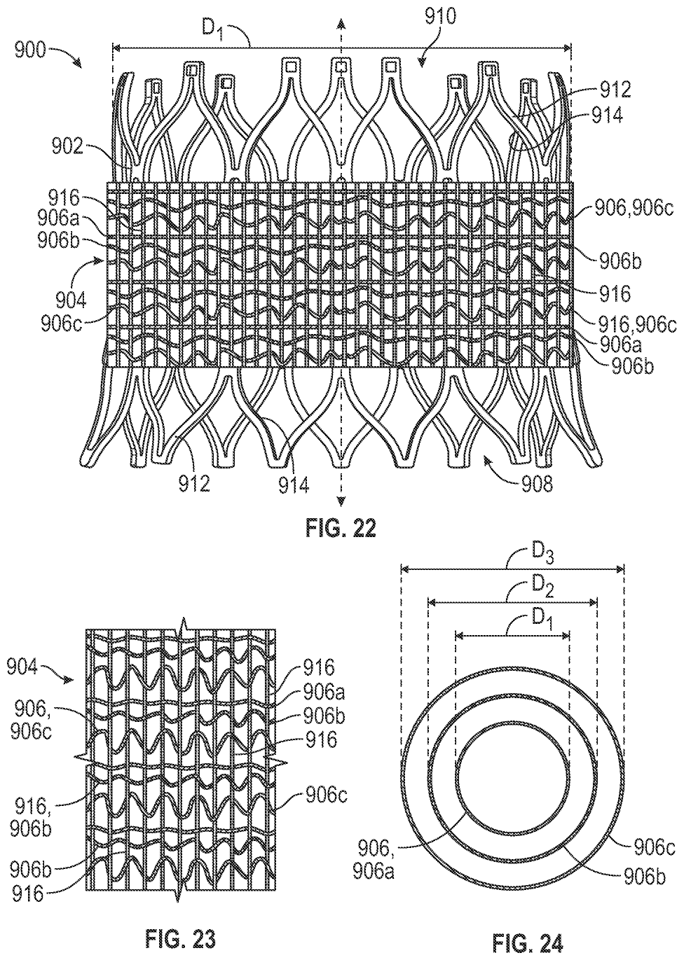

FIG. 22 illustrates a prosthetic valve 900 having a frame 902 and a skirt 904. The skirt 904 can be an annular body comprising a plurality of restriction filaments 906 configured (e.g., sized) to prevent the expansion of the prosthetic valve 900 past a certain diameter unless a force above a selected threshold is applied. Though the illustrated prosthetic valve 900 is a self-expanding prosthetic valve, it should be understood that such skirts 904 can be used with any type of prosthetic valve, including mechanical valves such as disclosed in International Application No. PCT/US2021/052745 and U.S. Pat. No. 10,603,165, balloon-expandable prosthetic heart valves, such as disclosed in U.S. Pat. Nos. 9,393,110 and 11,096,781, and self-expandable prosthetic heart valves, such as disclosed in U.S. Pat. No. 10,098,734.

The prosthetic valve 900 has an inflow end portion 908 and an outflow end portion 910. The prosthetic valve 900 can further a valvular structure (e.g., similar to valvular structure 18) and, in some examples, one or more additional skirts such as an inner skirt (e.g., similar to inner skirt 718 of FIG. 17), though these components are omitted for purposes of illustration. The frame 902 can comprise a plurality of struts 912 coupled together in an open lattice pattern defining a plurality of cells 914.

As shown in FIG. 22, in the illustrated example, the skirt 904 is an outer skirt extending around a circumference of the frame 902 and disposed on a radially outer surface of the frame. In other examples, the skirt 904 can be an inner skirt disposed on a radially inner surface of the frame 902, or a separately attached component coupled to the frame. In some examples, the skirt 904 can be coupled to the frame 902 using one or more sutures. In some examples, the skirt 904 can be woven through the cells 914 of the frame in an in-and-out (e.g., sinusoidal or woven) pattern. In the illustrated example, the skirt 904 is shown disposed around a central portion of the frame 902 (e.g., between the inflow end portion and the outflow end portion). However, in other examples, the skirt 904 can be disposed around the inflow portion 908 and/or the outflow portion 910 of the frame or can extend from the inflow end portion 908 of the frame to the outflow end portion 910. In some examples, the skirt 904 can extend over the inflow and/or outflow edge of the frame 902 such that the skirt is disposed at least partially on both the radially outer surface and the radially inner surface of the frame. In still other examples, the frame 902 can comprise two or more skirts 904 disposed at different locations on or within the frame 902.

The skirt 904 can be configured as a woven material or fabric comprising a plurality of filaments 916. One or more of the filaments 916 can be configured as restriction bands or restriction filaments 906. The restriction filaments 906 can have selected maximum diameters configured such that a physician can select the appropriate maximum diameter for the prosthetic heart valve 900 based on the specific anatomical configuration of the patient. The maximum diameters can be, for example, within the working range of a prosthetic heart valve such as between about 26 mm to about 29 mm.

In some examples, each restriction filament 906 can be a frangible restriction filament configured to tear, rupture, or break when a selected force threshold is exceeded. In other words, each restriction filament 906 can restrict radial expansion of the prosthetic valve 900 to a selected diameter unless and until a force above a selected threshold is applied to the filament 906. In some examples, the frangible restriction filaments can comprise a weakened portion or section such that when the restriction filament 906 is torn or ruptured, the break occurs at a selected location. In other examples, the restriction filaments 906 can be non-frangible restriction filaments and a physician can cut one or more restriction filaments prior to implantation such that the prosthetic valve expands to a preselected size, such as described with respect to the example of FIGS. 18-19.

The illustrated example shows three restriction filaments 906a, 906b, and 906c, each having a different maximum diameter D₁, D₂, and D₃, respectively, as shown in FIG. 24. However, in other examples, a skirt can comprise any number of restriction filaments 906 having various maximum diameters. As shown in FIG. 24, in the illustrated examples, a first filament 906a can be configured to restrict the diameter of the frame to a first expansion diameter D₁ corresponding with the maximum diameter of the first filament 906a, such as 27 mm. If expansion force(s) above a first selected threshold are applied to the frame 902 after the frame has reached the first expansion diameter D₁, the first filament 906a can break or rupture, allowing the frame 902 to expand to a second expansion diameter D₂ corresponding with the maximum diameter of the second filament 906b, such as 28 mm. If further expansion is desired, expansion force(s) above a second selected threshold can be applied to the frame 902 until the second filament 906b breaks or ruptures, allowing the frame 902 to expand to a third expansion diameter D₃ corresponding to the maximum diameter of the third filament 906c, such as 29 mm. If further expansion is desired, expansion force(s) above a third selected threshold can be applied to the frame until the third filament breaks or ruptures. In some examples, each selected threshold of force can be equal. In other examples, one or more of the selected force thresholds can be greater or lesser than the other selected force thresholds. For example, the threshold of force to break the first restriction filament 906a can be lesser than the threshold of force to break the second restriction filament 906b, which can be lesser than the threshold of force to break the third restriction filament 906c, or vice versa.

As shown in FIG. 23, the restriction filaments 906 can be woven into the material (e.g., fabric) of the skirt 904 (e.g., using an over and under weave pattern or a sinusoidal pattern). Embedding the restriction filaments 906 within the skirt 904 advantageously retains the torn or ruptured filaments 906 within the weave of the skirt fabric. Accordingly, the filaments 906 are not exposed to the bloodstream and prevent or minimize thrombosis or embolization. In some examples, the restriction filaments 906 can be woven in a wavy or serpentine pattern or otherwise woven in a slackened state such that the filaments 906 are retained within the fabric but are not under tension until the prosthetic valve reaches a respective filament's maximum diameter. For example, FIG. 22 shows the prosthetic valve 900 expanded to the first expansion diameter D₁ such that the first restriction filament 906a is substantially ring-shaped or circular and placed in tension while the other restriction filaments 906b, 906c are still in a slackened state. When frame 902 is in a radially compressed state for delivery into a patient's body, all of the restriction filaments 906a, 906b, 906c are in a slackened state with the first restriction filament 906a having the least amount of the slack, the second restriction filament 906b having more slack than the first restriction filament 906a, and the third restriction filament 906c having more slack than the second restriction filament 906b.

In other examples, the restriction filaments 906 can be separate from the weave of the skirt 904 and can be attached to the skirt 904 using, for example, one or more sutures.

In some particular examples, the restriction filaments 906 can be polyurethane (PU) and/or silicon filaments embedded within a skirt 904 comprising polyethylene (PE) fabric (e.g., a PE skirt). The skirt 904 can be retained in a relatively non-tensioned state, and the filaments embedded therein can comprise different lengths, allowing them to be highly stretchable.

In some examples, such as the illustrated example, the skirt 904 can comprise multiple restriction filaments 906 of each maximum diameter. Restriction filaments 906 having the same maximum diameter can be referred to as a ‘set of restriction filaments’ or as a ‘filament group.’ Though the illustrated example shows three filament groups 906a, 906b, and 906c, in other examples, the skirt 904 can comprise a greater or fewer number of filament groups.

As shown in FIGS. 22-23, in some examples, restriction filaments 906 belonging to the same set or group can be arranged in a repeating pattern with restriction filaments of other groups. For example, the pattern can repeat along the height of the skirt 904. In the illustrated example, the skirt 904 comprises a repeating pattern consisting of a first restriction filament 906a, a second restriction filament 906b, and a third restriction filament 906c. However, in other examples, for example, all restriction filaments 906 of a selected filament group can be positioned adjacent one another, or restriction filaments 906 of various filament groups can be disposed within the skirt at random, or in different patterns than the one shown in FIGS. 22-23 (e.g., filaments 906c can be disposed between filaments 906a and 906b, and/or filaments 906a can be disposed between filaments 906b and 906c, etc.).

In some particular examples, specific patterns of restriction filaments 906 can be used to cause the frame 902 to define one or more different shapes at different points in the expansion process. For example, a first group of restriction filaments 906a having a maximum diameter D₁ smaller than the other filament groups can be disposed at an inflow end portion 908 of the frame 902, such that the prosthetic valve 900 can define a V-shape unless and until the first filament group 906a is torn or ruptured, at which point the frame 902 can define a cylindrical shape, or other shape defined by another filament group. In other examples, such a technique can be used to define an A-shape or a barrel shape for the frame 902.

In some examples, the restriction filaments 906 can provide tactile feedback to the physician during the implantation process. For example, the physician may be able to feel (e.g., via the handle of the delivery device) when the frame 902 contacts a restriction filament 906 and/or when the restriction filament 906 is broken.

In some examples, the prosthetic valve 900 can be a self-expanding prosthetic valve having a maximum diameter greater than a diameter of the native annulus in which the prosthetic valve will be implanted. In such examples, the prosthetic valve 900 can be deployed to a diameter less than the maximum shape-set diameter of the prosthetic valve 900 during implantation (e.g., such that prosthetic valve 900 does not fully expand and such that at least one restriction filament 906 or filament group remains intact). Accordingly, if the native annulus loses tension over time, the remaining restriction filament(s) 906 prevent or mitigate expansion of the prosthetic valve 900 beyond the maximum diameter of the selected restriction filaments. Such a configuration advantageously mitigates the risk of delayed coronary obstruction (DCO) by preventing expansion of the prosthetic valve 900 past the selected diameter even if the force applied by the native annulus lessens. The force applied by the restriction filaments 906 of the skirt 904 at any of the selected diameters can be greater than the radial expansion force of the self-expandable valve.

In some examples, the prosthetic valve 900 can be configured to self-expand to a selected diameter less than the maximum diameter of any of the restriction filaments 906. The restriction filaments 906 can be configured to resist breaking under the expansion force of the frame exerted on the skirt through self-expansion of the frame. In such examples, actuators (such as actuators 200 described previously) can be used to increase the expansion force of the frame exerted on the skirt to break one or more of the restriction filaments and further expand the frame. As expansion forces are applied to the prosthetic valve 900 by the actuators 200, the sets of restriction filaments can be configured to tear or rupture as a selected threshold of radial force is exceeded, allowing the prosthetic valve 900 to incrementally expand to greater diameters.

In an exemplary method of implanting the prosthetic valve 900, a physician can mount the prosthetic valve 900 to the distal end portion of a delivery apparatus (such as delivery apparatus 600, described previously). The distal end portion of the delivery apparatus (along with the prosthetic valve 900) can be advanced through the patient's vasculature toward a selected implantation location (e.g., the native aortic valve). Once the prosthetic heart valve 900 is positioned at the selected location (such as within the native aortic annulus), the prosthetic heart valve can be deployed, for example, by withdrawing a delivery sheath or capsule (e.g., the distal end of portion 616 of shaft 606).

Once released from the delivery sheath, the prosthetic valve 900 can self-expand until it reaches a first diameter D₁ defined by the first set of restriction filaments 906a, at which point the prosthetic valve 900 is restrained from further expansion. The physician can then evaluate the fit of the prosthetic valve 900 within the native annulus. If further expansion of the prosthetic valve is required, the actuators (e.g., actuators 200) can be used to apply expansion force(s) to the frame 902 until a predetermined threshold of force is exceeded, causing the first set of filaments 906a to break and expanding or allowing the prosthetic valve 900 to expand to a second diameter D₂ defined by the second set of restriction filaments 906b. The physician can then re-evaluate the fit of the prosthetic valve within the annulus. This process can be repeated as necessary until the prosthetic valve 900 is expanded to a diameter that best fits the native annulus. For example, the prosthetic valve 900 can be expanded to a diameter sufficient to anchor the prosthetic valve in place against the surrounding tissue with minimal or no paravalvular leakage and without over-expanding and rupturing the native annulus.

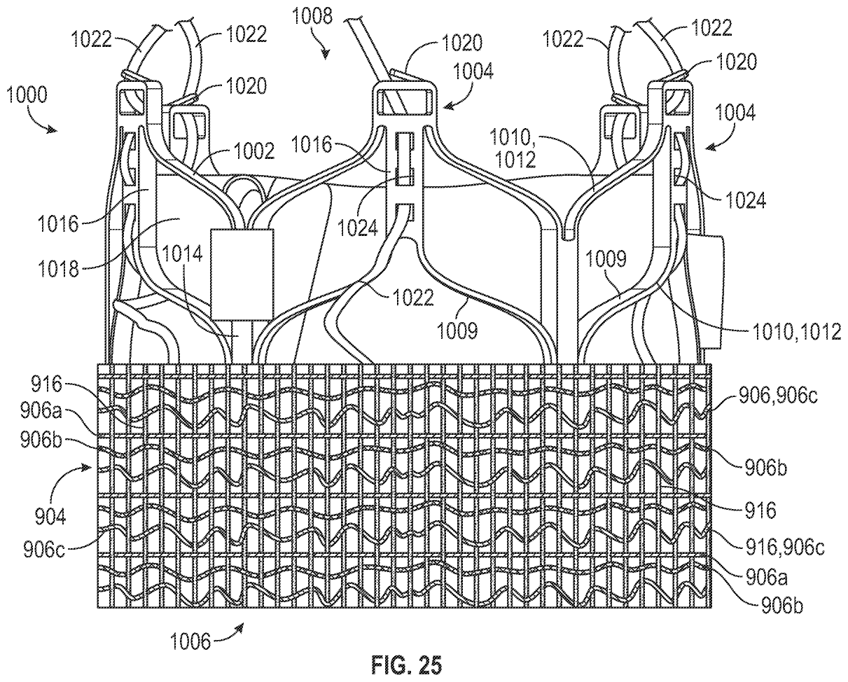

Referring to FIG. 25, in some examples, in lieu of prosthetic valve 900, the skirt 904 can be used with a prosthetic heart valve 1000 that is both is both self-expanding and/or mechanically expandable. For example, prosthetic valve 1000 can comprise a frame 1002 configured to self-expand from a radially compressed delivery state to at least a partially radially expanded state, and can comprise expansion mechanisms or actuator members 1004 that can be actuated to further radially expand the prosthetic valve 1000 to the fully radially expanded state. In another example, the prosthetic valve 1000 can be configured to self-expand to the fully expanded configuration and the actuators 1004 can be actuated to simply lock the prosthetic valve 1000 in the fully expanded state.

When configured to be self-expanding, the frame 1002 of the prosthetic valve 1000 can be constructed from a shape-memory material (e.g., Nitinol) that biases the frame 1002 towards a radially expanded state. That is, the prosthetic valve 1000 can be shape set in a partially radially expanded state or the fully radially expanded state (e.g., not the radially compressed state) so that the prosthetic valve 1000 is biased to return to the shape set radially expanded state when released from a restraining mechanism (e.g., a skirt, lasso, sheath, etc.). Further details of such self-expanding/mechanically-expandable prosthetic valves can be found, for example, in International Publication No. WO2022232010A1, which is incorporated by reference herein in its entirety.

The frame 1002 can be a radially expandable and compressible annular frame including an inflow end portion 1006 and an outflow end portion 1008, and one or more expansion and locking mechanisms or actuators 1004 configured to radially expand the frame 1002 and/or to lock the frame 1002 at one or more diameters to prevent radial compression of the prosthetic valve 1000. The frame 1002 can comprise a plurality of struts 1010 defining a plurality of windows or openings 1009. The struts 1010 can include rows of angled struts 1012 that extend between a first set of vertical struts or posts 1014 and a second set of vertical struts or posts 1016. The first and second sets of vertical struts 1014, 1016 can extend axially between the outflow end 1008 and the inflow end 1006 of the prosthetic valve 1000. The first set of vertical struts or posts 1014 can be configured to at least partially form, support, and/or define one or more commissures of the valvular assembly 1018 of the prosthetic valve 1000. The second set of vertical struts or posts 1016 can be configured to at least partially form, support, and/or define the expansion and locking mechanisms 1004. In this way, the one or more expansion and locking mechanisms 1004 can be at least partially integrated/incorporated into the frame 1002, thereby reducing the diameter and/or crimp profile of the frame 1002 when radially compressed (i.e., crimped).

The prosthetic valve 1002 further comprises a valvular assembly 1018 including one or more leaflets (e.g., three). The leaflets are configured to selectively open and close to regulate the flow of blood through the prosthetic valve 1000.

As mentioned, the prosthetic heart valve 1000 can comprise one or more expansion and locking mechanisms or actuators 1004 configured to at least partially expand the prosthetic valve 1000 and/or to lock the prosthetic valve 1000 in a radially expanded state. In the illustrated example, the prosthetic valve 1000 includes six expansion and locking mechanisms 1004. However, in other examples, the prosthetic valve 1000 can include a greater or fewer number of expansion and locking mechanisms 1004.

Each expansion and locking mechanism 1004 can comprise a locking element 1020 and an actuator member 1022. The actuator member 1022 can be coupled to the frame at first location (e.g., at or adjacent the inflow end of the frame) and at a second location via the locking element (which can be at or adjacent outflow end of the frame), the second location being axially spaced from the first location. The actuator member 1022 is fixedly secured to the frame at the first location and slidably coupled to the locking element 1020 at the second location such that actuator member can be pulled in a proximal direction relative to the locking element 1020 to cause radial expansion of the frame 1000 or to lock the frame in the radially expanded state, e.g., if the frame is self-expandable. As shown, the vertical struts 1016 can comprise one or more openings 1024 through which the actuator member 1022 can extend (e.g., in an in-and-out woven pattern) to facilitate coupling of the actuator member 1022 to the first and/or second locations.

The locking element 1020 (e.g., a pivoting or deformable spring tooth) is configured to hold/lock the prosthetic valve 1000 in a radially expanded state to prevent radial compression of the frame 1002 (e.g., by frictionally engaging the actuator member 1022). The locking element 1020 can be configured to allow movement of the actuator member 1022 relative to the locking element 1020 in a first direction (e.g., toward the outflow end portion 1008 of the frame or proximally) and prevent movement of the actuator member 1022 relative to the locking element 1020 in a second direction (e.g., toward the inflow end of the frame 1006 or distally).

The actuator member 1022 can be configured as a flexible tension member, such as a suture (such as a Dyneema® suture), string, cord, wire, cable, or other similar device that can be actuated by a physician (e.g., using the delivery apparatus) to radially expand the prosthetic valve 1000.

As mentioned, the prosthetic valve 1000 can comprise a skirt 904, as described previously. In some examples, as shown in FIG. 25, the skirt 904 can be configured as an outer skirt disposed on a radially outer surface of the frame 1002. For example, the outer skirt 904 can be sutured to the angled struts 1010, the vertical struts 1014, 1016, and/or to an inner skirt (not shown) positioned on the opposite surface of the frame.

In some examples, the prosthetic valve 1000 can have a maximum diameter to which it can self-expand that is less than or equal to the diameter D₁ of the first restriction filament 906a (e.g., such that restriction filament 906a is placed in tension). In such examples, once the prosthetic valve 1000 has been released from the delivery sheath of the delivery apparatus, the prosthetic valve 1000 can self-expand to the first diameter D₁. The physician can then actuate the expansion and locking mechanisms 1004 (e.g., by applying a pulling force to the actuator member(s) 1022 using the delivery apparatus) to apply expansion force(s) to the frame 1002 until a predetermined threshold of force is exceeded to break the first restriction filament 906a. The physician can then reduce the amount of force applied by the expansion and locking mechanisms 1004 and use the expansion and locking mechanisms 1004 to slowly expand the frame 1002 to a second diameter D₂ as defined by the second restriction filament 906b. At the second diameter D₂, the second restriction filament 906b can provide tactile feedback to the physician, alerting the physician that the prosthetic valve 1000 has reached the second diameter D₂. The physician can then re-evaluate the fit of the prosthetic valve within the annulus. If further expansion of the prosthetic valve is desired, the physician can actuate the expansion and locking mechanisms 1004 (e.g., by applying a pulling force to the actuator member(s) 1022 using the delivery apparatus) to increase the expansion force(s) until a second predetermined threshold is exceeded, causing the second restriction filament 906b to break. Once the second filament 906b is broken, the physician can once again reduce the expansion force applied by the expansion and locking mechanisms 1004 (e.g., by reducing the pulling force applied by the delivery apparatus) to slowly expand the frame 1002 to the next diameter. This process can be repeated as necessary for each restriction filament until the prosthetic valve 1000 is expanded to a diameter that best fits the native annulus.

In other examples, the prosthetic heart valve 1000 can be fully self-expandable. In such examples, the restriction filaments 906 can be configured such that a force greater than the self-expansion force of the frame 1002 is required to break the filaments. Such a configuration allows the prosthetic valve 1000 to self-expand in incremental stages. For example, upon release from a delivery sheath or capsule (e.g., such as the distal end portion 616 of shaft 606 described previously), the prosthetic valve 1000 can self-expand until it reaches a first diameter D₁ defined by the first set of restriction filaments 906a, at which point the prosthetic valve 1000 is restrained from further expansion by the first set of filaments 906a. The physician can then evaluate the fit of the prosthetic valve 1000 within the native annulus. If further expansion of the prosthetic valve is required, the expansion and locking mechanisms 1004 can be used to apply expansion force(s) to the frame 1002 until a predetermined threshold of force is exceeded, causing the first set of filaments 906a to break. With the first filaments 906a thus broken, the prosthetic valve 1000 can self-expand to a second diameter D₂ defined by the second set of restriction filaments 906b. The physician can then re-evaluate the fit of the prosthetic valve 1000 within the annulus. This process can be repeated as necessary until the prosthetic valve 1000 is expanded to a diameter that best fits the native annulus. For example, the prosthetic valve 1000 can be expanded to a diameter sufficient to anchor the prosthetic valve in place against the surrounding tissue with minimal or no paravalvular leakage and without over-expanding and rupturing the native annulus.

In still other examples, the prosthetic heart valve 1000 can be fully mechanically expandable. In such examples, the force used to expand the frame 1002 can be less than the force used to break the restriction filaments 906. For example, the physician can apply a first force (e.g., an expansion force) to expand the frame to a first diameter D₁ defined by the first restriction filament 906a. In some examples, the physician can receive tactile feedback (e.g., via the handle) notifying the physician that the frame has reached the first diameter D₁. The physician can then evaluate the fit of the prosthetic valve 1000 within the native annulus. If further expansion of the prosthetic valve is required, a second force (e.g., a breaking force) can be applied to the frame 1002 until a predetermined threshold of force is exceeded, causing the first filament 906a to break. The physician can then apply the first force to expand the frame to the second diameter D₂. This process can be repeated as necessary until the prosthetic valve 1000 has been expanded to the desired diameter.

As mentioned, the actuator members 1022 can be coupled to a control device or handle (e.g., handle 604 described previously) configured to actuate the actuator members 1022 in order to radially expand and/or radially collapse the frame 1002. In some examples, the handle can comprise one or more mechanisms configured to limit the forces applied to the actuator members 1022 (e.g., limiting the forces to one or more selected force magnitudes) instead of relying on the physician to apply a specified force magnitude using skill. For example, the handle can include a force selecting mechanism such as a knob or other actuator configured to allow the physician to select the amount/magnitude of force applied when the actuator members 1022 are actuated. In such examples, the force-selecting mechanism can have, for example, a first setting or level configured to apply a first force to expand the frame 1002, and a second setting or level configured to apply a second force to break one or more restriction filaments 906. The first force can be sufficient to expand the frame 1002 while being insufficient to cause breakage of the restriction filaments 906.

The forces can be applied mechanically (e.g., the physician can set a level and then mechanically actuate the actuator members) or electronically (e.g., the physician can select a level and actuate a button/actuator such that the handle automatically applies the selected force to the actuator members via one or more motors). A force-selecting mechanism can advantageously mitigate or prevent the physician from unintentionally breaking/rupturing the restriction filaments by applying too much force. This can prevent inadvertent over-expansion of the prosthetic heart valve within the native annulus.

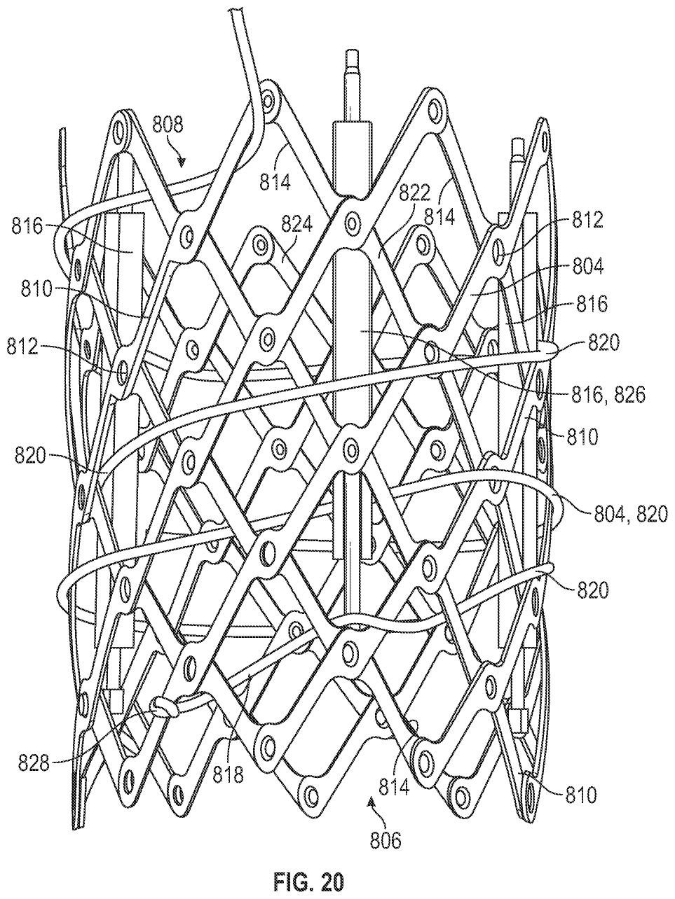

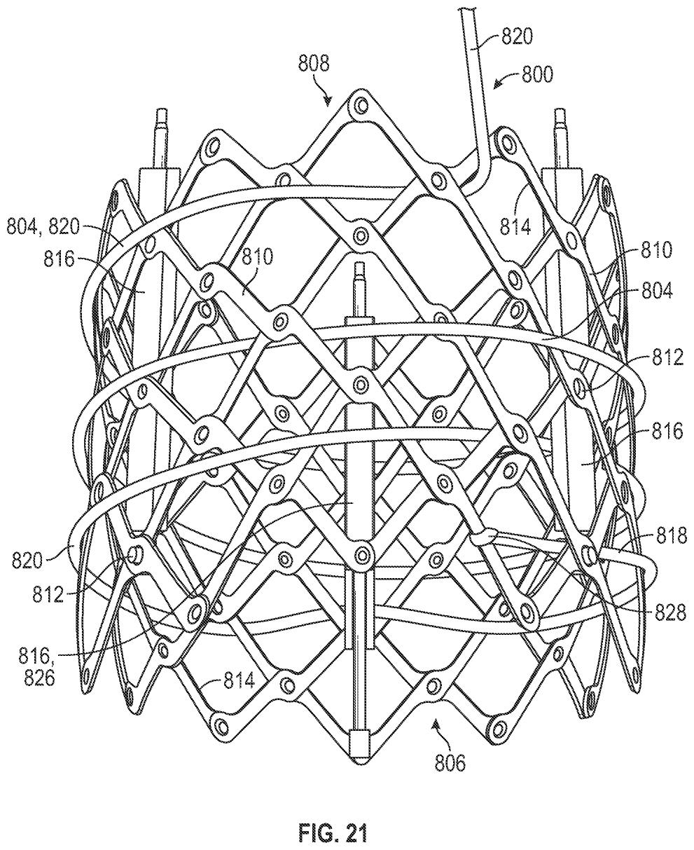

Referring now to FIGS. 20-21, in lieu or in addition to a valve belt such as valve belts 304, 400, or 500, or skirt 904, a prosthetic valve 800 having a frame 802 can comprise a flexible tension member 804 configured to allow controllable gradual valve expansion. The tension member 804 can apply a radially inwardly directed force to the frame 802 that can be gradually lessened such that the frame can expand at a controlled rate (e.g., a rate selected by the physician) to a selected diameter.

Though the illustrated prosthetic valve 800 is a mechanically-expandable prosthetic valve, it should be understood that the tension members 804 described herein can be used with any type of prosthetic valve, including mechanical valves such as those disclosed in International Application No. PCT/US2021/052745 and U.S. Pat. No. 10,603,165, balloon-expandable prosthetic heart valves, such as disclosed in U.S. Pat. Nos. 9,393,110 and 11,096,781, and self-expandable prosthetic heart valves, such as disclosed in U.S. Pat. No. 10,098,734.

Prosthetic valve 800 comprises a frame 802 having an inflow end portion 806 and an outflow end portion 808, and can include a valvular structure (e.g., valvular structure 18) and inner and/or outer skirts, as previously described, through these components are omitted for purposes of illustration. The frame 802 can comprise a plurality of struts 810 coupled together at junctions 812 in a lattice pattern defining a plurality of cells 814. The prosthetic valve 800 can have a self-expanding range, e.g., the frame 802 can inherently self-expand from a fully crimped diameter to a partially expanded diameter, and a mechanically expandable range, e.g., the frame 802 can be mechanically expanded from the partially expanded diameter to a fully expanded diameter. The prosthetic valve 800 can comprise one or more expansion and locking mechanisms 816 (e.g., three) configured to mechanically expand the frame 802 from the partially expanded diameter to the fully expanded diameter and to lock the frame 802 in the radially expanded configuration.

As shown in FIG. 20, the flexible tension member 804 can have a first end portion 818 releasably coupled to the frame 802 at a first location and a second end portion 820 coupled to the handle of the delivery apparatus (e.g., handle 604 of apparatus 600 shown in FIG. 15). The handle 604 can include a knob or other actuation mechanism operatively coupled to the tension member 804 to apply a force (e.g., a proximally directed force) to the tension member 804. In the illustrated example, the first end portion 818 is coupled to the frame at a location adjacent the inflow end portion 806. However, in other examples, the first end portion 818 can be coupled to the frame 802 at any axial location along the length of the frame and at any location circumferentially around the frame 802.

The tension member 804 can extend at least partially around the circumference of the frame 802. For example, the tension member 804 can be disposed such that it extends around the frame 802 in a continuous helical manner defining a plurality of loops 820. In in illustrated example, the tension member 804 extends helically around the frame 802 such that it defines three loops 820. However, in other examples, the tension member 804 can define a greater or fewer number of loops, for example, a single loop. In still other examples, the tension member 804 can be disposed such that it spans a distance less than the full circumference of the frame 802.

As shown in FIG. 20, in some examples, the tension member 804 can be coupled to the frame 802 by weaving the tension member through selected cells 814 of the frame 802 in an in-and-out manner. At points, the tension member 804 can extend over the radially outer surface 822 of the frame, and at other points the tension member 804 can extend over the radially inner surface 824 of the frame. In some examples, portions of the tension member 804 can pass between the radially inner surface 824 of the frame and a radially outer surface 826 of one or more expansion and locking mechanisms 816. In other examples, the tension member 804 can be coupled to the frame 802 at selected attachment points by, for example, passing the tension member 804 through one or more eyelets and/or openings formed in the struts 810 and/or at the junctions 812 between the struts 810, etc.

The tension member 804 can be, for example, a suture (e.g., a single filament suture or a multi-filament suture), a flexible wire (e.g., a metal wire formed from stainless steel, Nitinol or other suitable metals), a cable (e.g., a braided cable formed from metal or polymeric strands) or any other similar materials that can be threaded through the frame 802 and placed in tension to radially compress the prosthetic valve as described herein.

In the illustrated example, the first end portion 818 of the tension member 804 is releasably coupled to the frame 802 using one or more knots 828. However, in other examples, the tension member 804 can be releasably coupled to the frame using one or more clips, hooks, and/or other such releasable mechanisms. In some examples, an optional sheath or tube can extend over the second end portion 820 of the tension member 804.

In some examples, the tension member 804 can pass through a locking component coupled to the frame 802. The locking component can be configured to retain the tension member 804 at a selected tension thereby retaining the frame 802 at a selected diameter. Such a configuration can advantageously prevent or mitigate further spontaneous radial expansion of the prosthetic valve.

The tension member 804 can be used to gradually allow expansion of the prosthetic valve 800 in the following exemplary manner. The prosthetic valve 800 can be connected a delivery apparatus, such as delivery apparatus 600 described previously. The distal end portion of the delivery apparatus 600 (along with prosthetic valve 800) can be advanced through the vasculature of a patient to a selected implantation site.

Once at the implantation site, the distal end portion 616 of the shaft 606 covering the prosthetic valve 800 can be retracted, and the tension member 804 can be tensioned (e.g., using the handle 604) to prevent inherent expansion caused by the natural resiliency of the frame 802.

The tension member 804 can continue to be tensioned as one or more expansion forces are applied to the frame (e.g., using expansion and locking mechanisms 816). The tension in tension member 804 can be gradually released, allowing the frame to expand gradually (e.g., at a controlled rate) to a larger diameter and/or its fully expanded diameter. Such a configuration can advantageously prevent or mitigate unintended radial expansion of the prosthetic valve (e.g., caused by inherent frame expansion) and/or control expansion to prevent radial “jumps” caused by, for example, stepped expansion mechanisms (e.g., expansion and locking mechanisms that utilize a ratchet system), thereby maximizing the physician's control over positioning the prosthetic valve.

In some examples, once the prosthetic valve 800 has been expanded, the tension in the tension member 804 can be fully released and the tension member 804 can be uncoupled from the frame 802. The delivery apparatus 600 can be released from the prosthetic valve 800 and removed from the body. In other examples, such as those comprising a locking component, the tension member 804 can be disconnected from the delivery apparatus 600 and can remain in the patient's body with the prosthetic valve 800.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A skirt for an implantable prosthetic device, comprising:
an annular fabric matrix radially expandable from a radially compressed configuration to a first diameter upon application of a radially outwardly directed force via the implantable prosthetic device; and
a plurality of frangible restriction filaments embedded in the fabric matrix, each restriction filament having a selected maximum diameter different from that of at least one of the other restriction filaments, and each restriction filament being configured to break when a radially outwardly directed force applied to the restriction filament exceeds a predetermined threshold.

Example 2. The skirt of any example herein, particularly example 1, wherein a first frangible restriction filament of the plurality of frangible restriction filaments is configured to break when the radially outwardly directed force exceeds a first predetermined threshold to allow radial expansion of the annular fabric matrix to a second diameter.

Example 3. The skirt of any example herein, particularly any one of examples 1-2, wherein a second frangible restriction filament of the plurality of restriction filaments is configured to break when the radially outwardly directed force exceeds a second predetermined threshold to allow radial expansion of the annular fabric matrix to a third diameter.

Example 4. The skirt of any example herein, particularly any one of examples 1-3, wherein the plurality of frangible restriction filaments comprise at least one of polyurethane (PU) and silicon.

Example 5. The skirt of any example herein, particularly any one of examples 1-4, wherein the annular fabric matrix comprises woven polyethylene (PE) fabric.

Example 6. The skirt of any example herein, particularly any one of examples 1-5, wherein the frangible restriction filaments remain embedded within the fabric matrix after breaking.

Example 7. The skirt of any example herein, particularly any one of examples 1-6, wherein the frangible restriction filaments are woven into the fabric matrix in an in-and-out pattern.

Example 8. The skirt of any example herein, particularly any one of examples 1-7, wherein the predetermined threshold of force of each restriction filament is the same.

Example 9. The skirt of any example herein, particularly any one of examples 1-8, wherein the frangible restriction filaments are woven are woven in the fabric matrix such that each frangible restriction filament is in a slackened state when a diameter of the fabric matrix is less than the maximum diameter of the frangible restriction filament.

Example 10. A skirt for an implantable prosthetic device, comprising:
an annular body configured to extend around a circumference of the prosthetic device;
one or more sets of frangible restriction filaments coupled to the annular body;
wherein the annular body is radially expandable from a radially compressed configuration to a first diameter upon application of a radially outwardly directed force via the implantable prosthetic device;
wherein a first set of frangible restriction filaments of the one or more sets of frangible restriction filaments is configured to break when the radially outwardly directed force exceeds a first predetermined threshold to allow radial expansion of the annular body to a second diameter; and
wherein the one or more set of frangible restriction filaments remain embedded within the annular body when broken.

Example 11. The skirt of any example herein, particularly example 10, wherein a second set of frangible restriction filaments of the one or more sets of restriction filaments is configured to break when the radially outwardly directed force exceeds a second predetermined threshold to allow radial expansion of the annular body to a third diameter.

Example 12. The skirt of any example herein, particularly any one of examples 10-11, wherein the plurality of frangible restriction filaments comprise at least one of polyurethane (PU) and silicon.

Example 13. The skirt of any example herein, particularly any one of examples 10-12, wherein the annular body comprises woven polyethylene (PE) fabric.

Example 14. The skirt of any example herein, particularly any one of examples 10-13, wherein the sets of frangible restriction filaments are coupled to the annular body by weaving the sets of frangible restriction filaments into a weave of the annular body.

Example 15. The skirt of any example herein, particularly any one of examples 10-14, wherein the sets of frangible restriction filaments are coupled to the annular body using one or more sutures.

Example 16. The skirt of any example herein, particularly any one of examples 10-15, wherein each set of frangible restriction filaments has a selected maximum diameter different from the selected maximum diameters of the other sets of frangible restriction filaments.

Example 17. The skirt of any example herein, particularly any one of examples 10-16, wherein the skirt comprises first, second, and third sets of frangible restriction filaments.

Example 18. The skirt any example herein, particularly any one of examples 17, wherein the first set of frangible restriction filaments has a maximum diameter less than a maximum diameter of the second set of frangible restriction filaments, and wherein the third set of restriction filaments has a maximum diameter greater than the maximum diameters of the first and second sets.

Example 19. The skirt of any example herein, particularly any one of examples 10-18, wherein the sets of frangible restriction filaments are disposed such that the filaments define a repeating pattern.

Example 20. An assembly, comprising:
an implantable prosthetic device comprising a frame movable between a radially compressed configuration and a radially expanded configuration; and
a skirt extending circumferentially around the frame, the skirt comprising:

an annular fabric matrix radially expandable from a radially compressed configuration to a first diameter upon application of a radially outwardly directed force via the implantable prosthetic device, and
at least one first frangible restriction filament and at least one second frangible restriction filament woven in the fabric matrix, wherein the first frangible restriction filament has a first maximum diameter and the second frangible restriction filament has a second maximum diameter that is greater than the first maximum diameter;
wherein the first frangible restriction filament is configured to break when the frame is expanded to the first maximum diameter and applies a force to the first frangible restriction filament sufficient to break the first frangible restriction filament; and
wherein the second frangible restriction filament is configured to break when the frame is expanded to the second maximum diameter and applies a force to the second frangible restriction filament sufficient to break the second frangible restriction filament.

Example 21. The assembly of any example herein, particularly example 20, wherein the implantable prosthetic device is at least partially self-expandable.

Example 22. The assembly of any example herein, particularly any one of examples 20-21, wherein the prosthetic device is a prosthetic heart valve comprising a plurality of leaflets that regulate a flow of blood through the frame.

Example 23. The assembly of any example herein, particularly any one of examples 20-22, wherein the implantable prosthetic device comprises one or more actuators configured to apply one or more expansion forces to the implantable prosthetic device.

Example 24. The assembly of any example herein, particularly example 23, wherein the first frangible restriction filament is configured to break when the force applied by the frame exceeds a first predetermined threshold to allow further radial expansion of the implantable prosthetic device to the second maximum diameter.

Example 25. The assembly of any example herein, particularly example 24, wherein the second frangible restriction filament is configured to break when the force applied by the frame exceeds a second predetermined threshold to allow radial expansion of the implantable prosthetic device to a third diameter.

Example 26. The assembly of any example herein, particularly any one of examples 20-25, wherein the frangible restriction filaments remain woven within the fabric matrix when broken.

Example 27. The assembly of any example herein, particularly any one of examples 20-26, wherein the frangible restriction filaments are woven into the fabric matrix in a sinusoidal pattern.

Example 28. The assembly of any example herein, particularly any one of examples 20-27, wherein the skirt is disposed on a radially outer surface of the implantable prosthetic device.

Example 29. The assembly of any example herein, particularly any one of examples 20-28, wherein the skirt is disposed on a radially inner surface of the implantable prosthetic device.

Example 30. The assembly of any example herein, particularly any one of examples 20-29, wherein the first frangible restriction filament is configured to have a slackened state when a diameter of the frame is less than the first maximum diameter and a tensioned state when the diameter of the frame is equal to the first maximum diameter, and wherein the second frangible restriction filament is configured to have a slackened state when the diameter of the frame is less than the second maximum diameter and a tensioned state when the diameter of the frame is equal to the second maximum diameter.

Example 31. The assembly of any example herein, particularly any one of examples 20-30, wherein the first frangible restriction filament comprises a plurality of first frangible restriction filaments.

Example 32. The assembly of any example herein, particularly any one of examples 20-31, wherein the second frangible restriction filament comprises a plurality of second frangible restriction filaments.

Example 33. The assembly of any example herein, particularly example 32, wherein the plurality of first frangible restriction filaments the plurality of second frangible restriction filaments are arranged in the fabric matrix in a pattern comprising alternating first and second frangible restriction filaments along a height of the fabric matrix.

Example 34. A method, comprising:
advancing an implantable prosthetic device comprising a skirt through a patient's vasculature to a selected implantation site, the skirt extending around a circumference of the prosthetic device and comprising an annular body and first and second restriction filaments embedded within the annular body;
radially expanding the prosthetic device and the skirt to a first diameter defined by the first restriction filament;
applying a first expansion force greater than a first predetermined threshold to the prosthetic device to break the first restriction filament; and
radially expanding the prosthetic device and the skirt to a second diameter defined by the second restriction filament.

Example 35. The method of any example herein, particularly example 34, further comprising applying a second expansion force greater than a second predetermined threshold to the prosthetic device to break the second restriction filament; and radially expanding the prosthetic device and the skirt to a third diameter defined by a third restriction filament.

Example 36. The method of any example herein, particularly any one of examples 34-35, wherein the skirt further comprises a plurality of additional first, second, and third restriction filaments arranged in a repeating pattern.

Example 37. The method of any example herein, particularly any one of examples 34-36, wherein radially expanding the prosthetic device and the skirt to the first diameter comprises allowing the prosthetic device to self-expand to the first diameter.

Example 38. The method of any example herein, particularly any one of examples 34-37, wherein the skirt comprises a woven fabric and wherein the restriction filaments are embedded within the skirt by weaving them into the fabric.

In view of the many possible examples to which the principles of the disclosure may be applied, it should be recognized that the illustrated examples are only preferred examples and should not be taken as limiting the scope of the disclosure. Rather, the scope of the disclosure is defined by the following claims. We therefore claim all that comes within the scope and spirit of these claims.

We claim:

1. A skirt for an implantable prosthetic device, comprising:
an annular fabric matrix radially expandable from a radially compressed configuration to a first diameter upon application of a radially outwardly directed force via the implantable prosthetic device; and
a plurality of frangible restriction filaments embedded in the fabric matrix, each restriction filament having a selected maximum diameter different from that of at least one of the other restriction filaments, and each restriction filament being configured to break when a radially outwardly directed force applied to the restriction filament exceeds a predetermined threshold.

2. The skirt of claim 1, wherein a first frangible restriction filament of the plurality of frangible restriction filaments is configured to break when the radially outwardly directed force exceeds a first predetermined threshold to allow radial expansion of the annular fabric matrix to a second diameter.

3. The skirt of claim 2, wherein a second frangible restriction filament of the plurality of restriction filaments is configured to break when the radially outwardly directed force exceeds a second predetermined threshold to allow radial expansion of the annular fabric matrix to a third diameter.

4. The skirt of claim 1, wherein the plurality of frangible restriction filaments comprise at least one of polyurethane (PU) and silicon.

5. The skirt of claim 1, wherein the annular fabric matrix comprises woven polyethylene (PE) fabric.

6. The skirt of claim 1, wherein the frangible restriction filaments remain embedded within the fabric matrix after breaking.

7. The skirt of claim 1, wherein the frangible restriction filaments are woven into the fabric matrix in an in-and-out pattern.

8. The skirt of claim 1, wherein the predetermined threshold of force of each restriction filament of the plurality of frangible restriction filaments is the same.

9. The skirt of claim 1, wherein the plurality of frangible restriction filaments are woven in the fabric matrix such that each frangible restriction filament is in a slackened state when a diameter of the fabric matrix is less than the maximum diameter of the frangible restriction filament.

10. A skirt for an implantable prosthetic device, comprising:
an annular body configured to extend around a circumference of the prosthetic device;
one or more sets of frangible restriction filaments coupled to the annular body;
wherein the annular body is radially expandable from a radially compressed configuration to a first diameter upon application of a radially outwardly directed force via the implantable prosthetic device;
wherein a first set of frangible restriction filaments of the one or more sets of frangible restriction filaments is configured to break when the radially outwardly directed force exceeds a first predetermined threshold to allow radial expansion of the annular body to a second diameter; and
wherein the one or more set of frangible restriction filaments remain embedded within the annular body when broken.

11. The skirt of claim 10, wherein a second set of frangible restriction filaments of the one or more sets of restriction filaments is configured to break when the radially outwardly directed force exceeds a second predetermined threshold to allow radial expansion of the annular body to a third diameter.

12. The skirt of claim 10, wherein the one or more sets of frangible restriction filaments comprise at least one of polyurethane (PU) and silicon.

13. The skirt of claim 10, wherein the annular body comprises woven polyethylene (PE) fabric.

14. The skirt of claim 10, wherein the sets of frangible restriction filaments are coupled to the annular body by weaving the sets of frangible restriction filaments into a weave of the annular body.

15. The skirt of claim 10, wherein the sets of frangible restriction filaments are coupled to the annular body using one or more sutures.

16. The skirt of claim 10, wherein each set of frangible restriction filaments has a selected maximum diameter different from the selected maximum diameters of the other sets of frangible restriction filaments.

17. The skirt of claim 10, wherein the skirt comprises first, second, and third sets of frangible restriction filaments.

18. The skirt of claim 17, wherein the first set of frangible restriction filaments has a maximum diameter less than a maximum diameter of the second set of frangible restriction filaments, and wherein the third set of restriction filaments has a maximum diameter greater than the maximum diameters of the first and second sets.

19. The skirt of claim 10, wherein the sets of frangible restriction filaments are disposed such that the filaments define a repeating pattern.
